(19) United States
(12) Patent Application Publication
(10) Pub. No.: US 2014/0336709 A1
(43) Pub. Date: Nov. 13, 2014

(54) MULTI-THREADED PEDICLE SCREW SYSTEM

(71) Applicant: BAXANO SURGICAL, INC., Raleign, NC (US)

(72) Inventors: Eugene E. Avidano, Wilmington, NC (US); Walter Scott Hill, Southport, NC (US); Brandon B. Arthurs, Wilmington, NC (US)

(21) Appl. No.: 14/208,884

(22) Filed: Mar. 13, 2014

Related U.S. Application Data
(60) Provisional application No. 61/851,773, filed on Mar. 13, 2013.
(60) Provisional application No. 61/852,949, filed on Mar. 25, 2013.

(51) Int. Cl. A61B 17/70; A61B 17/86; A61B 17/00

(52) U.S. Cl. CPC A61B 17/7032; A61B 17/7085; A61B 17/00234; A61B 17/8615; A61B 2017/564
USPC 606/271; 606/279; 606/308

(57) ABSTRACT

A pedicle screw system includes a multi-threaded bone fastener configured with a constant pitch and a placement tower assembly for delivery of the bone fastener.

The present disclosure claims the benefit of a pair of co-pending and commonly assigned United States Provisional Applications, specifically, U.S. Provisional Application No. 61/851,773 filed Mar. 13, 2013 for Pedicle Screw Device and Implantation System and U.S. Provisional Application No. 61/852,949 filed Mar. 25, 2013 for Multi-Threaded Pedicle Screw System. Both of these two provisional applications are incorporated in their entirety into this disclosure by reference herein.

### FIELD OF THE DISCLOSURE

The present invention relates to medical devices, systems and methods used in minimally invasive spinal surgery. More particularly, this invention is directed to spinal stabilization by mechanically fixing a posterior portion of a spine, and in particular bone fasteners, such as pedicle screws.

### BACKGROUND

The spine is formed of a series of bones called vertebrae. A vertebra consists of two essential parts including an anterior segment or body, and a posterior part, or vertebral or neural arch. These two parts enclose the vertebral foramen, which together form a canal for the protection of the spinal cord. The vertebral arch consists of a pair of pedicles and a pair of laminae. The body is the largest part of a vertebra, and is generally cylindrical with flattened upper and lower surfaces. The pedicles are two short, thick processes, which project backward, one on either side, from the upper part of the body, at the junction of its posterior and lateral surfaces.

Instability of spinal joints may result from, for example, trauma (to ligamentous structures; fracture, or dislocation); degenerative disease processes (e.g., rheumatoid arthritis; degenerative spondylosis; spondylolisthesis; spinal stenosis); tumor; infection, or congenital malformation that may lead to significant pathological translation, or longitudinal displacement. Cord compression and trauma to the spinal cord can result in respiratory distress, pain, nerve dysfunction, paresis and paralysis, or even sudden death. Therefore, the need for spinal stabilization in the setting of pathological instability is paramount.

Spinal arthrodesis, or fusion, provides needed biomechanical stability and is a therapy used to treat such instability. The objective is to create a stable biomechanical environment and provide the biological requirements for osseous fusion. Adequate decompression of the neurological structures, where indicated, and recreation of normal sagittal and coronal alignment are prerequisites prior to an arthrodesis procedure. Spinal fixation has been achieved using a variety of techniques to provide stabilization and/or spinal alignment, followed by fusion, or arthrodesis by means of bone graft insertion. Over the years, various techniques and systems have been developed for correcting spinal injuries and/or degenerative spinal processes.

Thus, spinal correction frequently requires stabilizing a portion of the spine to facilitate fusing portions of the spine or other correction methodologies and medical correction of this type is frequently employed for many spinal conditions, such as, for example, degenerative disc disease, scoliosis, spinal stenosis, or the like. Frequently, these corrections also require the use of implants and/or bone grafts. Stabilizing the spine allows bone growth between vertebral bodies such that a portion of the spine is fused into a solitary unit.

Several techniques and systems have been developed for correcting and stabilizing the spine and facilitating fusion at various levels of the spine. In one type of system, a rod is disposed longitudinally along the length of the spine in the region of concern and engages various vertebrae along its length. The rod engages, or more typically a pair of generally parallel rods engage the spine using fixation elements, such as anchors, attached to vertebral bodies by a bone screw that is inserted into the pedicle and penetrates into the body of the vertebra.

Anatomy and correction frequently require aligning the rod and screw at various angles along the length of the portion of correction. In order to provide this alignment, polyaxial screws/anchors have been developed. Generally, the bone screw spinal fixation systems are installed using placement towers which removably clamp the screw housing thereby allowing manipulation of the bone screw system from outside the body. After placement of the bone screws and housing as required, the placement towers can be removed.

While stabilization procedures, and in particular posterior fusion surgical implants, instrumentation, and techniques, continue to evolve in the pursuit of improvements in clinical outcomes (e.g., the highest fusion rate with the shortest time to fusion and improvement in neurological function), and in simplicity of use, notwithstanding, there remains a need for ongoing advancements in bone screw configurations and constructs leading to progress in the surgical management of complex spinal disorders, to accommodate an increased spectrum of anatomical variations, to enable simplicity of instrumentation placement, and to avoid certain adverse events such as loss of spinal alignment, in order to achieve more rigid stabilization in a wider variety of spinal diseases.

More particularly, rod and screw constructs known in the art have limitations, e.g., experience failures (loosening, breakage, or cutout), including rod failure (breakage or telescoping) or screw failure (breakage, migration or pullout). Moreover, in addition to the need to overcome problems of screw loosening, there exists a need for systems for spinal stabilization which do not obscure the surgeon's view as a screw is being inserted, and where construct profiles maximize space for graft material, and in which the components are configured to permit greater flexibility in deployment by the surgeon to achieve optimum fit.

### GENERAL COMMENTS AND TERMINOLOGY

In the context of the present disclosure, as used herein the terms “assembly” or “constructs” are sometimes used interchangeably and refer to implants, implant systems, instruments, or instruments systems which are configured to comprise multiple components, which may or may not be contiguous. It is further understood that individual components may themselves be configured as sub-assemblies, e.g., comprising a plurality of component materials, and that the formation of the components may involve intermediate processes or appliances.

It will also be understood that upon formation of assemblies from multiple components and deployment, individual components of the present disclosure may or may not remain as discernibly distinct. It will also be understood that, for convenience, system components may be packaged and provided either individually, or as in “kits,” and either as reusable or disposable.

As used herein, the term “biocompatible” refers to an absence of chronic inflammation response or cytotoxicity when or if physiological tissues are in contact with, or exposed to (e.g., wear debris) the materials and devices of the present disclosure. In addition to biocompatibility, in another aspect of the present disclosure it is preferred that the materials comprising the implant and instrument systems are sterilizable.

In one aspect of the present disclosure, certain components of the device assemblies and systems of the present disclosure are configured to comprise biocompatible materials and are able to withstand, without wear, multiple cycles/procedures without failing. For example, materials selected may include but are not limited to, biomedical titanium, cobalt-chromium, or medical grade stainless steel alloys.

It will be further understood that the length and dimensions of implant components and instruments described herein will depend in part on the target site selection of the treatment procedure and the physical characteristics of the patient, as well as the construction materials and intended functionality, as will be apparent to those of skill in the art

In order to make it easier for a reader to find certain sections of this document that are of particular interest to the reader, a series of headings have been used. These headings are solely for the purpose of helping readers navigate the document and do not serve to limit the relevance of any particular section exclusively to the topic listed in the heading.

In the context of this discussion:

Anterior refers to “in front” of the spinal column;

Ventral and posterior refers to “behind” the column (dorsal);

Cephalad means towards the patient's head;

Caudal refers to the direction or location that is closer to the feet;

Proximal is closer to the surgeon;

Distal is in use more distant from the surgeon;

Superior refers to a top or front surface, and

Inferior refers to a back or bottom surface of a device.

When referencing tools,

distal would be the end intended for insertion into the patient and

proximal refers to the other end, generally the end closer to, e.g., a handle for the tool and the user.

The sequence of operations (or steps) is not limited to the order presented in the claims or figures unless specifically indicated otherwise.

As used herein, it will be understood that the terms rod, spinal rod, longitudinal rod, are sometimes used interchangeably and refer to devices within the stabilization construct that connect and align the vertebrae. It will also be understood that as used herein, the terms bone fastener, screw, pedicle screw and polyaxial screw are sometimes used interchangeably and refer to devices adapted to receive or connect to a spinal rod, or adapted to further align and secure a construct of screw and rod to part of the spine to be stabilized.

### SUMMARY OF THE DISCLOSURE

There are described and disclosed herein spinal stabilization and fixation systems including multi-threaded bone fasteners and placement tower assemblies. The bone fastener (bone screw) may be deployed with the use of the placement tower assembly. After a fusing rod is placed through a housing holding the head of the bone screw, a locking cap may be used to lock the polyaxial position.

Teachings of the present disclosure include a pedicle screw having:

a shank comprising an elongate shaft extending along a screw axis, wherein the shank includes a proximal portion and a distal portion; a male first thread located on the external surface of the shank extending from the proximal portion to the distal portion, wherein the first thread is configured such that rotation of the screw in a first direction advances the screw into bone, a male second thread located on the external surface of the shank extending from the proximal portion to the distal portion, wherein the second thread is configured such that rotation of the screw in a first direction advances the screw into bone, the second thread being offset approximately 180 degrees from the first thread; a head attached to the proximal portion of the shank, wherein the head comprises a male head thread configured to accept a female threaded collar, wherein rotation of the collar in a second direction, opposite the first direction, advances the collar towards the shank and couples the collar to the head; wherein the first thread and second thread have constant pitch, and a substantially constant major diameter and minor diameter and wherein the first thread and second thread are continuous and uninterrupted and wherein the first thread and the second thread start substantially the same distance from the head and end substantially the same distance from the head.

Teachings of this disclosure include a pedicle screw having a shank comprising an elongate shaft extending along a screw axis, wherein the shank comprises a proximal portion and a distal portion; a male first thread located on the external surface of the shank extending from the proximal portion to the distal portion, wherein the first thread is configured such that rotation of the screw in a first direction advances the screw into bone a male second thread located on the external surface of the shank extending from the proximal portion to the distal portion; wherein the second thread is configured such that rotation of the screw in a first direction advances the screw into bone, a male third thread located on the external surface of the shank extending from the proximal portion towards the distal portion; wherein the third thread is configured such that rotation of the screw in a first direction advances the screw into bone, and a head attached to the proximal portion of the shank, wherein the head comprises a male head thread configured to accept a female threaded collar, wherein rotation of the collar in a second direction, opposite the first direction, advances the collar towards the shank and couples the collar to the head; wherein the first thread, second thread, and third thread have a proximal end substantially the same distance from the head, wherein the first thread, second thread have distal end at a distance from the head D but the third thread has a distal end that is a different distance from the head so that a proximal portion of the shank has three threads equally spaced apart and a distal portion of the shank with only two threads separated by a gaps of 120 and 240 degrees.

Teachings of the present disclosure include a bone screw with a shank and a head, the shank having a dual lead thread pattern with a first thread offset 180 degrees from a second thread with a handedness of the first thread equal to a handedness of the second thread; a minor diameter of the shank equal to D from a distal decreasing taper at a distal end of the shank which decreases the shank diameter to less than D to a proximal taper at a proximal end of the shank which increases the shank diameter to more than D; the head having a third thread of opposite handedness from the first thread and the second thread, the third thread engaged with a corresponding threads on a collar having a convex curved surface, a portion of the head distal to the third thread having a curve which acts as a continuation of the convex curved surface of the collar, and the head having a driver engagement section on a proximal end of the head.

Teachings of the present disclosure include a bone screw having:
a threaded shank including a distal end portion; and a proximal end portion;
said threaded shank defining a first threaded section extending from said distal end portion toward said proximal end portion;
said threaded shank defining a second threaded section extending contiguously from said first threaded section toward said proximal end portion and adapted for engagement in cortical bone;
said second threaded section comprising a finer thread pattern relative to the first threaded section;
wherein said threaded shank includes a first helical threading extending along said threaded shank from said first from said first threaded section and into said second threaded section;
said threaded shank including a second helical threading interleaved with said first helical threading to define said second threaded section for engagement with the cortical bone;
each of said first and second helical threadings having a substantially equal pitch; and
a head portion extending from said proximal end portion of said threaded shank;
said second threaded section having a third helical threading interleaved with said first helical threading and said second helical threading;
said third helical threading having a substantially equal pitch with the first helical threading and second helical threading;
an offset between the first helical threading and the second helical threading being equal to an offset between the second helical threading and the third helical threading and equal to an offset between the third helical threading and the first helical threading;
wherein the absence of the third helical threading in the first threaded section allows the bone screw to engage with cancellous bone to have a first helical bone volume located between a first side of the first helical threading and a first side of the second helical threading, the first helical bone volume having a first-inter threading width; and
a second helical bone volume located between the second side of the first helical threading and the second side of the second helical threading, the second helical bone volume having a second inter-threading width that is more than double the first inter-threading width;
to provide two different threading bone interactions for a section of cancellous bone while having a third threading-bone interaction in said second threaded section for use with cortical bone.

Teachings of the present disclosure include a tower assembly with:
a bone screw with a bone screw shank and a bone screw head, the bone screw shank having at least one shank thread of a first handedness;
the bone screw head having a head thread of opposite handedness from the shank thread, the head thread engaged with a corresponding thread on a collar having a convex curved surface, a portion of the bone screw head distal to the head thread having a curve which acts as a continuation of the convex curved surface of the collar; and
the bone screw head having a driver engagement section on a proximal end of the bone screw head;
the bone screw shank extending beyond a U-shaped housing through an opening in a distal end of the housing that is too small to allow the collar to pass through the hole;
the U-shaped housing adapted to allow a fusing rod to be passed through a pair of U-shaped openings before the locking cap is advanced distally to lock both the fusing rod from longitudinal movement and the housing from polyaxial movement relative to the bone screw head;
a delivery tower engaged with a proximal end of the housing via protrusions that extend distally from the tower to engage features on an outer perimeter of the housing;
a screw driving rod retainer within a longitudinal bore in the delivery tower that engages a set of threads inside the proximal end of the housing; and
a bone screw driver within a longitudinal bore in the screw driving rod retainer with a driver tip that extends to engage the driver engagement section of the proximal end of the bone screw.

Aspects of the teachings contained within this disclosure are addressed in subsequent claims submitted with this application upon filing Rather than adding redundant restatements of the contents of the claims, these claims should be considered incorporated by reference into this summary, although the present disclosure in not intended to be limited in scope by these initial claims.

This summary is meant to provide an introduction to the concepts that are disclosed within the specification without being an exhaustive list of the many teachings and variations upon those teachings that are provided in the extended discussion within this disclosure. Thus, the contents of this summary should not be used to limit the scope of the claims that follow.

Inventive concepts are illustrated in a series of examples, some examples showing more than one inventive concept. Individual inventive concepts can be implemented without implementing all details provided in a particular example. It is not necessary to provide examples of every possible combination of the inventive concepts provided below as one of skill in the art will recognize that inventive concepts illustrated in various examples can be combined together in order to address a specific application.

Other systems, methods, features and advantages of the disclosed teachings will be or will become apparent to one with skill in the art upon examination of the following figures and detailed description. It is intended that all such additional systems, methods, features and advantages be included within the scope of and be protected by the accompanying claims.

### BRIEF DESCRIPTION OF THE FIGURES

The accompanying figures, which are incorporated in and constitute part of this specification, are included to illustrate and provide a further understanding of the system and method of the invention. Together with the description, the figures serve to explain the principles of the invention. Unless indicated, the components in the figures are not necessarily to scale, emphasis instead being placed upon illustrating the principles of the invention. Moreover, in the figures, like referenced features designate corresponding parts throughout the different views.

FIG. 1 is a view of a bone screw and housing system.

FIG. 2 is a view of a pair of housing retaining blades.

FIG. 3 is a view of a one of the pair of twin housing retaining blades with installed translation pegged blades.

FIG. 4 is another view of a one of the pair of twin housing retaining blades with installed translation pegged blades.

FIG. 5 is a view of the pair of twin housing retaining blades with installed translation pegged blades.

FIG. 6 illustrates a locking collar that can be used with the examples described above.

FIG. 7 illustrates the locking collar of FIG. 6 in transparent view.

FIG. 8 illustrates the locking collar engaging the blade portion of the placement tower assembly.

FIG. 9 illustrates a blade pin.

FIG. 10 illustrates the blade pin engaged with other portions of the placement tower assembly.

FIG. 11 shows the placement tower assembly illustrated in FIG. 10 from approximately the top of the device.

FIG. 12 illustrates a portion of the engagement of the bone screw and housing system and one half of the placement tower assembly (for visualization purposes).

FIG. 13 shows the elements illustrated in FIG. 12 with the addition of a second translating pegged blade and another retaining pin and a retaining pin head.

FIG. 14 shows a transected partial view of the placement tower assembly engaging the bone screw and housing system.

FIG. 15 shows a second transected partial view of the placement tower assembly engaging the bone screw and housing system.

FIG. 16 is an enlarged view of FIG. 15 without all of the element lead lines to allow a less unobstructed view of the assembly.

FIG. 17 shows a transected partial view of the placement tower assembly engaging the bone screw and housing system with the fusing rod shown.

FIG. 18 illustrates a bone screw driving rod.

FIG. 19 further illustrates a bone screw driving rod 700

FIG. 20 illustrates screw driving rod retainer 800.

FIG. 21 further illustrates a screw driving rod retainer 800.

FIG. 22 illustrates the placement tower assembly holding the bone screw and housing system with the bone screw driving assembly.

FIG. 23 illustrates the persuader approximately halfway down the axial length of the placement tower assembly.

FIG. 24 illustrates the persuader tamping or persuading the fusing rod to seat fully.

FIG. 25 shows a locking cap driver that may be inserted through the placement tower assembly to tighten the locking cap.

FIG. 26 illustrates the placement tower assembly attached to the bone screw and housing system via the housing with select components removed.

FIG. 27 is a flow chart for the sequence of deployment.

FIG. 28 illustrates a side view of one example of a pedicle screw.

FIG. 29 illustrates a side view of another example of a pedicle screw.

FIG. 30 shows a top perspective view of bone screw.

FIG. 31 is a side view of the bone screw from FIG. 30.

FIG. 32 is a top view of the bone screw from FIG. 30.

FIG. 33 is a bottom view of the bone screw from FIG. 30.

### DETAILED DESCRIPTION

Described herein are examples directed towards a bone screw and an associated assembly, especially for application in the spinal stabilization arena. However, as can be appreciated, the bone screws and associated assemblies disclosed herein can be used in any of a number of clinical applications where insertion of a screw into bone is desired. The devices, systems, and methods described herein are not intended to limit the scope of this disclosure. Rather, it will be apparent to one of skill in the art that the devices, systems, and methods disclosed herein can be used in varied clinical applications. Additionally, it should be noted that elements of one example can be combined with elements of another example, except where the function of the components prohibits such combinations.

One of skill in the art will recognize that there is a close cooperation between the tower delivery system and the pedicle screw system. Thus, modifications to the pedicle screw system used will usually result in corresponding changes to the tower system used for delivery.

Examples of a bone screw system adapted for simple coupling and simple de-coupling to a placement tower include a bone screw as taught in the art in a housing with notches on the side as taught in the art as well as vertically disposed notches or holes in the top of the housing adapted to accept coupling pins from a placement tower. Examples of a placement tower system adapted for simple coupling and simple de-coupling to a bone screw system include a two-bladed system with coupling pins adapted to translate vertically into coupling holes or notches on the top of a bone screw system's housing to temporarily hold the two blades around the housing of the bone screw system

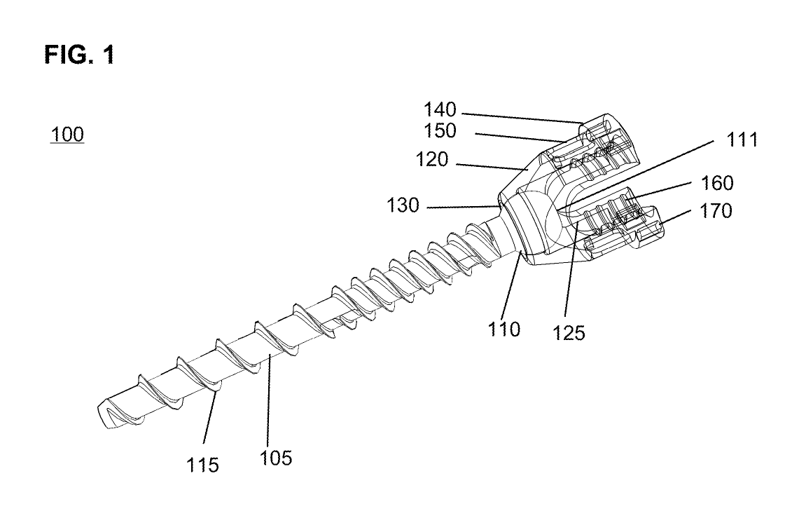

FIG. 1 illustrates a bone screw and housing system 100. The bone screw and housing system 100 can include a bone screw 105 which can include a bone screw head 110 with a bone screw driving insert portion 111 and one or more sets of bone screw threads 115. The bone screw and housing system 100 may include a housing 120 which has a housing hollow center channel 125, a housing lower opening 130, at least one housing upper flange 140, at least one housing clip accepting inset 150, housing internal threads 160 and a least one peg accepting slot 170.

As shown in FIG. 1, the bone screw 105 has a shaft with the bone screw threads 115. The bone screw has a bone screw head 110 at the proximal end. As discussed below, the bone screw head may be a removable component that engages with a set of threads on the proximal end of the bone screw 105. The bone screw 105 fits or slips down through the housing lower opening 130 in the housing 120. The housing lower opening 130 is at least slightly smaller than the largest diameter of the bone screw head 110. Therefore, the bone screw 105 can swivel while installed in the housing 120 as shown in FIG. 1.

In some examples, the housing lower opening 130 can be circular. In other examples, the housing lower opening 130 can be oval or have another shape. The housing lower opening 130 is preferably smaller than the bone screw head 110 in at least one dimension. In some examples, the housing lower opening 130 allows the bone screw 105 to swivel about a cone with a conical angle in the range of about 10 to 180 degrees, including the ranges of about 20 to 170 degrees, about 30 to 160 degrees, about 40 to 150 degrees, about 50 to 140 degrees, about 60 to 130 degrees, about 70 to 120 degrees, about 80 to 110 degrees, and about 90 to 100 degrees.

As shown in FIG. 1, the housing 120 of the illustrated arrangement includes a substantially hollow U-shaped retaining member. The housing 120 includes the housing lower opening 130 in its base out of which the bone screw 105 extends. As disclosed above, the housing lower opening 130 is smaller than the diameter of the bone screw head 110 in at least one dimension to thereby hold the bone screw head 110 from slipping out the bottom of the housing 120. The U-shape can be created by the housing hollow center channel 125 which allows visualization and subsequent insertion of a stabilization rod through the housing 120.

On the inside surface of the housing 120, there may be threads, which will be discussed in more depth below. On the outside surface of the housing 120 there may be include structures used for attachment or coupling to the application systems as will be discussed in more detail below. The structures on the outer surface of the housing 120 can include at least one housing clip accepting inset 150, the housing upper flange 140 and at least one peg accepting slot 170. The housing 120 of FIG. 1 illustrates two housing clip accepting insets 150, one on each side of the “U” and four peg accepting slots 170, two on each side of the U-shape, however, in other examples more there may be a different number of peg accepting slots (either more or less than four). The housing upper flange 140 can lie at the upper edge of the U-shape of the housing 120.

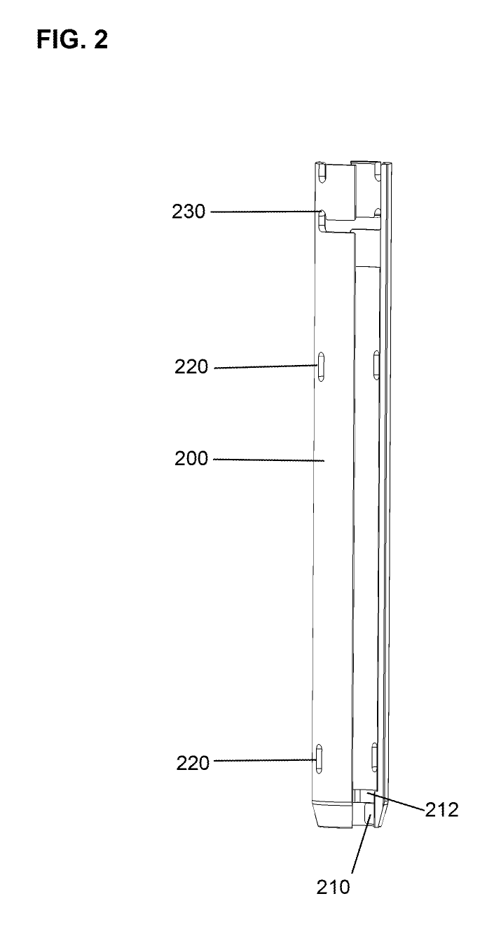

FIG. 2 illustrates a pair of housing retaining blades 200 from the placement tower assembly 1500 (shown absent the other portions of the placement tower assembly 1500 for illustration purposes only). The twin housing retaining blades 200 can include two housing retaining blades 200, at least one outset housing clip 210, at least one inset flange slot 212, at least one retaining pin slot 220, and at least one L-slot 230.

The twin housing retaining blades 200 can be semi-circular in shape. In some examples, each housing retaining blade 200 has an arch that may be the range of

about 40-170 degrees,

about 50-150 degrees,

about 60-130 degrees,

about 70-110 degrees, or

about 80-90 degrees.

The end of the twin housing retaining blades 200 including the outset housing clip 210 and inset flange slot 212 is configured to retain the housing 120 of the pedicle screw system.

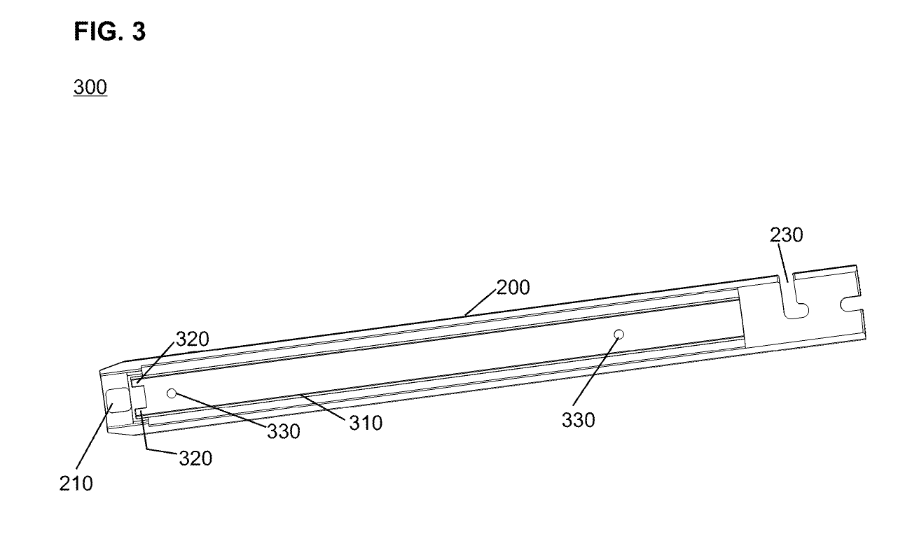

FIG. 3 illustrates a housing retaining assembly 300. The full placement tower assembly 1500 includes two housing retaining assemblies 300, but only one is shown in FIG. 3 to allow better visualization. The housing retaining assembly 300 includes the housing retaining blade 200, having the outset housing clip 210, the inset flange slot 212, and the L-slot 230 (as shown in FIG. 2), a translating pegged blade 310, at least one retaining pin 330 (there may be the same number of retaining pins 330 on the translating pegged blade 310 as there are retaining pin slots 220 on the housing retaining blade 200) and at least one housing retaining peg 320.

In operation, the outer surface of the translating pegged blade 310 is defined by the cylindrical inner surface of the housing retaining blade 200. The translating pegged blade 310 is held to the inner surface of the housing retaining blade 200 by using at least one retaining pin 330. FIG. 3 illustrates the translating pegged blade 310 which is held in place on the inner surface of the housing retaining blade 200 by using two retaining pins 330. By reference to FIG. 2, the retaining pin slots 220 are oblong in the direction of the cylindrical axis of the twin housing retaining blades 200. The at least one retaining pin 330 of the translating pegged blade 310 exits the at least one retaining pin slot 220 of housing retaining blade 200 and therefore allows the translating pegged blade 310 to translate vertically along the axis of the cylinder defined by the translating pegged blade 310 and the housing retaining blade 200.

In some examples, the translating pegged blade 310 includes only two housing retaining pegs 320 (as illustrated in FIG. 3). In other examples, the translating pegged blade 310 includes about four housing retaining pegs 320, about three housing retaining pegs 320, or only one housing retaining peg 320.

In some examples, the translating pegged blade 310 includes two retaining pins 330 (as shown in FIG. 3). In other examples, the translating pegged blade 310 includes about four retaining pins 330, about three retaining pins 330, or only one retaining pin 330.

In some examples, the translating pegged blade 310 is an arcuate segment comprising fewer degrees axially than the housing retaining blade 200. In other examples, the translating pegged blade 310 is an arcuate segment comprising the same degrees axially as the housing retaining blade 200. In yet other examples, the translating pegged blade 310 is and arcuate segment comprising more degrees axially than the housing retaining blade 200.

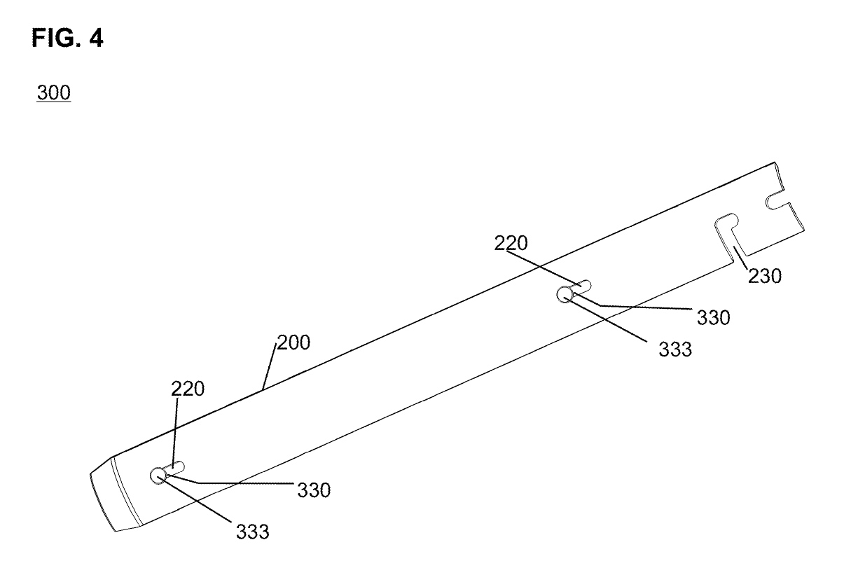

FIG. 4 illustrates the housing retaining assembly 300 of FIG. 3 from the outside of the assembly (the opposite side as is shown in FIG. 3). The housing retaining assembly 300 of FIG. 4 shows the housing retaining blade 200 which obscures the translating pegged blade 310. Also included in FIG. 4 are the at least one retaining pin slot 220, the at least one retaining pin 330, at least one retaining pin head 333 and the L-slot 230.

In operation, the translating pegged blade 310 (not shown in FIG. 4) is held to the inner concave surface of the housing retaining blade 200 by the at least one retaining pin 330 which extends through the at least one retaining pin slot 220 and terminates in the retaining pin head 333 which is larger in diameter than at least one aspect of the retaining pin slot 220. Therefore, the retaining pin head 333 allows the retaining pin 330 only vertical axial motion. One retaining pin head 333 is provided for each retaining pin 330. For example, if two retaining pins 330 are used there can be provided two retaining pin heads 333 (one on each of the two retaining pins 330).

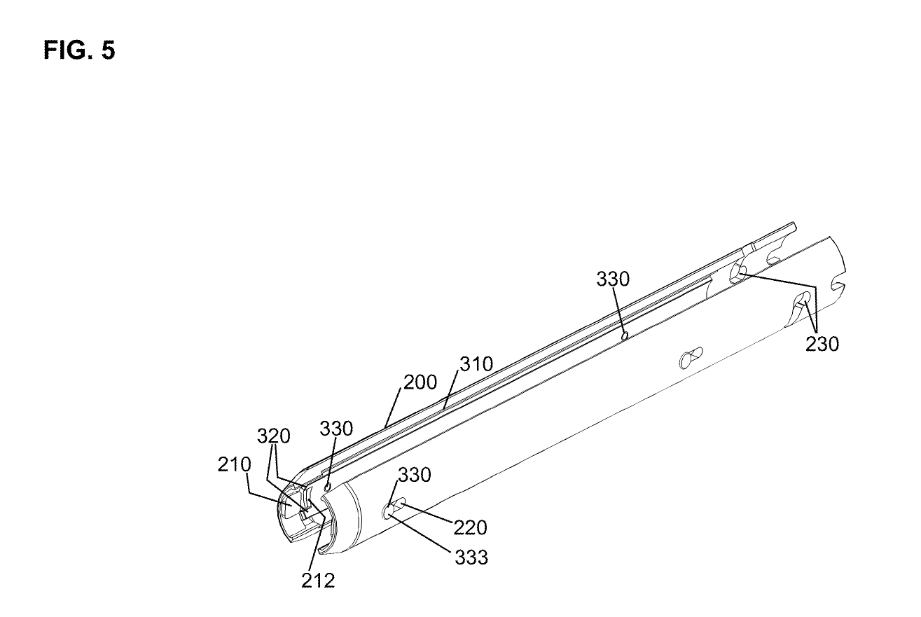

FIG. 5 illustrates the same structures and elements shown in FIG. 4, including both halves of the device.

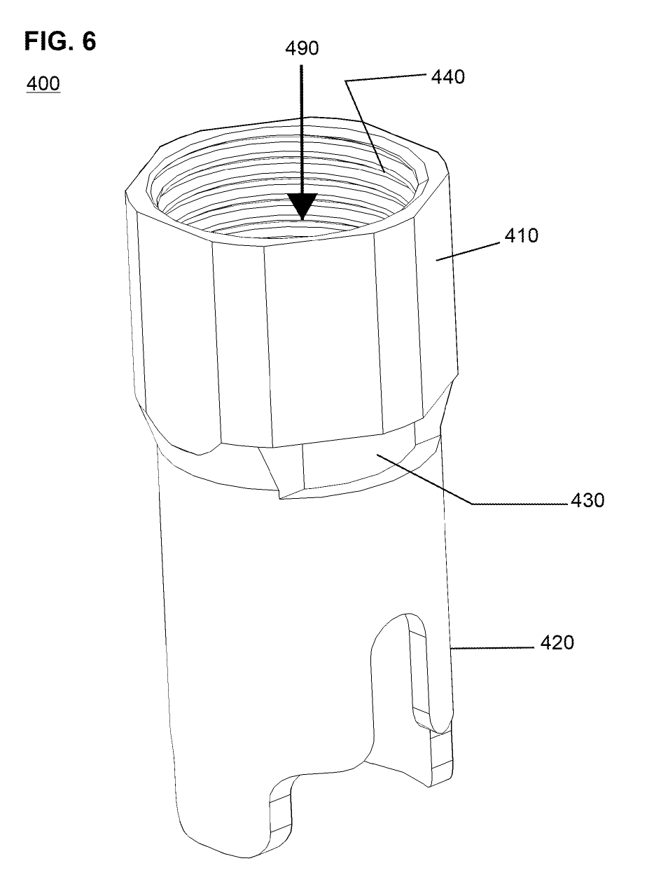

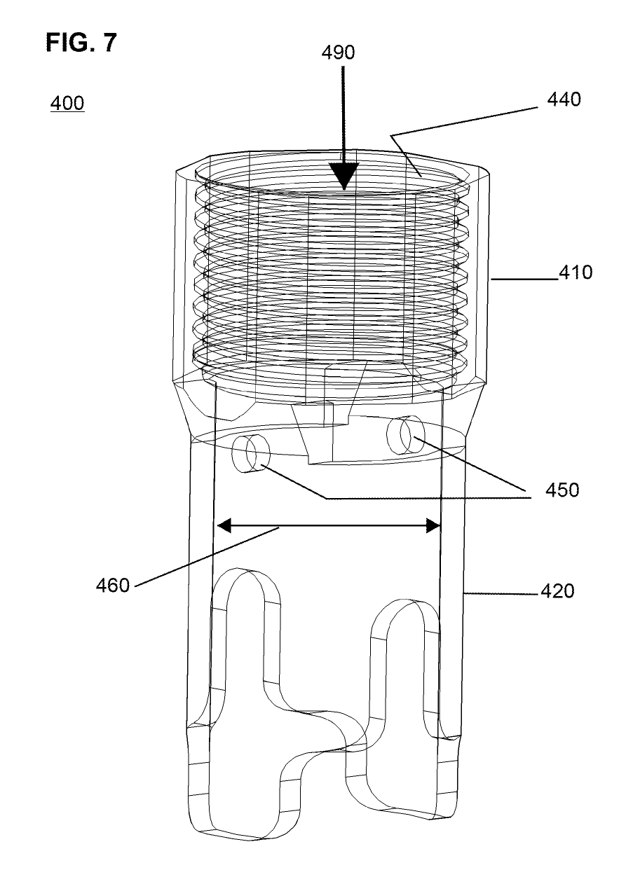

FIG. 6 illustrates a locking collar 400 that can be used with the examples described above. The locking collar 400 can include a locking collar neck 410, a locking collar lower sleeve 420, a collar window 430, locking collar threads 440, and a locking collar internal lumen 490. FIG. 7 illustrates the locking collar 400 of FIG. 6 in transparent view. The locking collar 400 of FIG. 7 also illustrates the at least one locking pin 450 and the locking collar internal diameter 460.

The internal lumen of the locking collar 400 (and the inner surface of the locking collar threads 440) defines a cylinder with a locking collar internal diameter 460 which is substantially equal to the cylinder defined by the outer surface of the twin housing retaining blades 200.

In some examples, there are two locking pins 450 located 180 degrees apart on the inner surface of the locking collar 400 disposed below the locking collar threads 440,. In other examples, there are multiple locking pins 450.

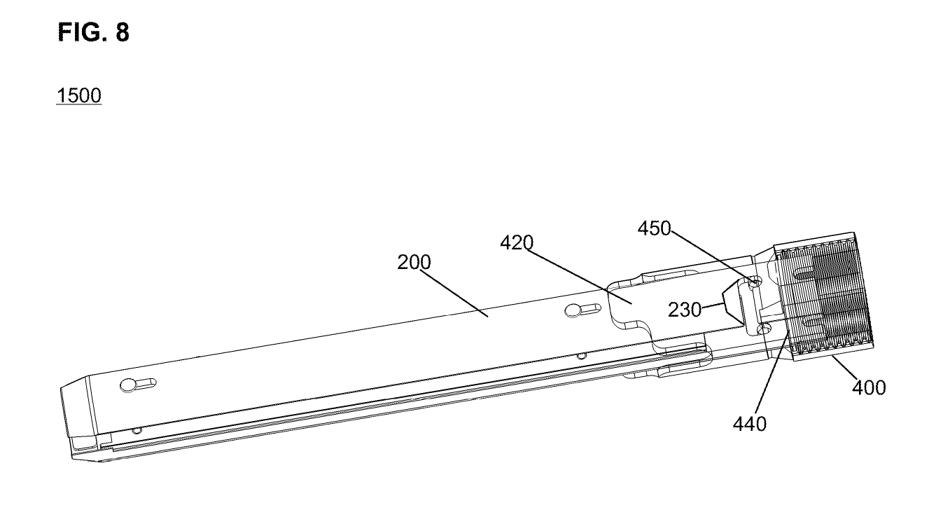

FIG. 8 illustrates the locking collar 400 engaging the blade portion of the placement tower assembly 1500. As can be seen from the transparent locking collar 400, the two locking pins 450 fit into the L-slots 230 of the twin housing retaining blades 200. Also, it can be seen that the inner surface of the locking collar lower sleeve 420 defines a cylinder approximately equal to the cylinder defined by the outer surface of the twin housing retaining blades 200.

In some examples, the twin housing retaining blades 200 terminate where the locking collar threads 440 terminate. In other examples, the twin housing retaining blades 200 terminate below where the locking collar threads 440 terminate.

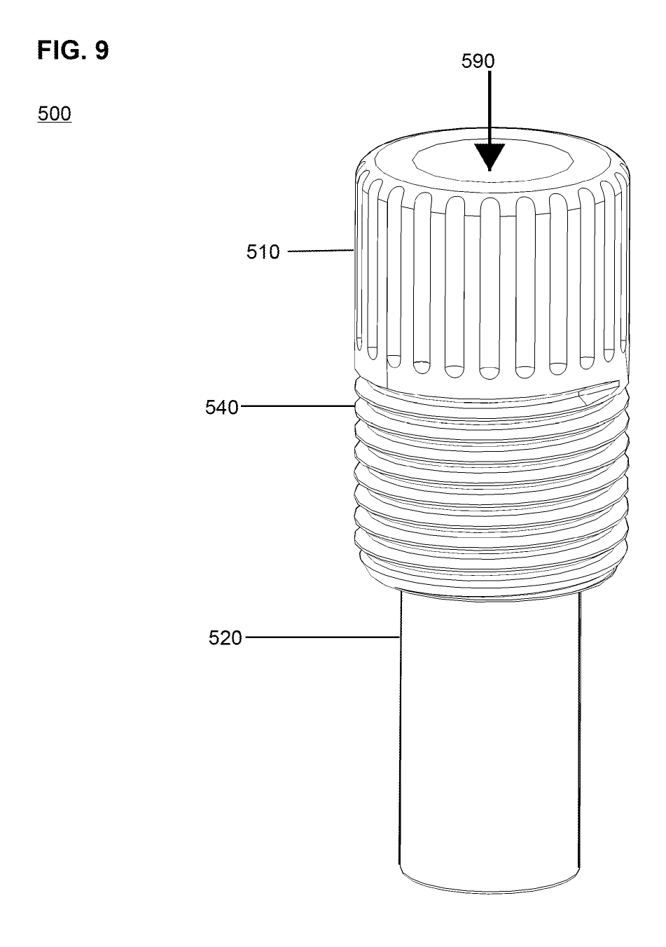

FIG. 9 illustrates a blade pin 500. The blade pin 500 includes a blade pin knob 510, a blade pin inner sleeve 520, blade pin threads 540 and a blade pin inner lumen 590.

The blade pin threads 540 are sized so as to mate with the locking collar threads 440 of the locking collar 400. By comparison to the inner surface of the locking collar 400 which defines a cylinder approximately equal to the cylinder defined by the outer surface of the twin housing retaining blades 200, the outer surface of the blade pin 500 defines a cylinder approximately equal to the cylinder defined by the inner surface of the twin housing retaining blades 200. Therefore, if the smaller cylinder (defined by the blade pin 500) were placed inside the larger cylinder (defined by the locking collar 400), the distance between the two cylinders would be approximately equal to the thickness of each housing retaining blade 200 of the twin housing retaining blades 200.

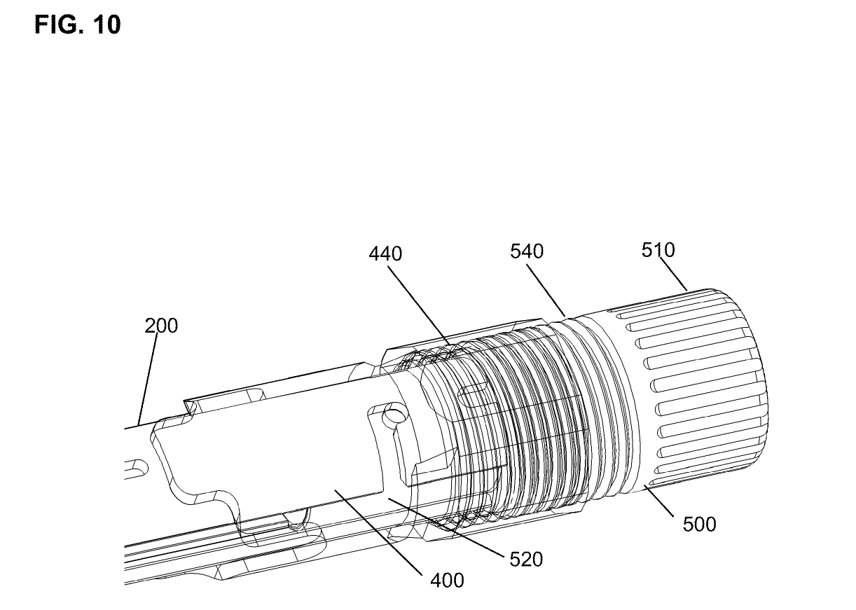

FIG. 10 illustrates the blade pin 500 engaged with other portions of the placement tower assembly 1500. In operation, the blade pin threads 540 of the blade pin 500 thread into the locking collar threads 440 of the locking collar 400. As the blade pin 500 threads into the locking collar 400 by turning the blade pin knob 510, it will hold the twin housing retaining blades 200 in place. As the blade pin inner sleeve 520 fits just inside the twin housing retaining blades 200, if the blade pin 500 is threaded in further, it will push down on the top of the translating pegged blade 310.

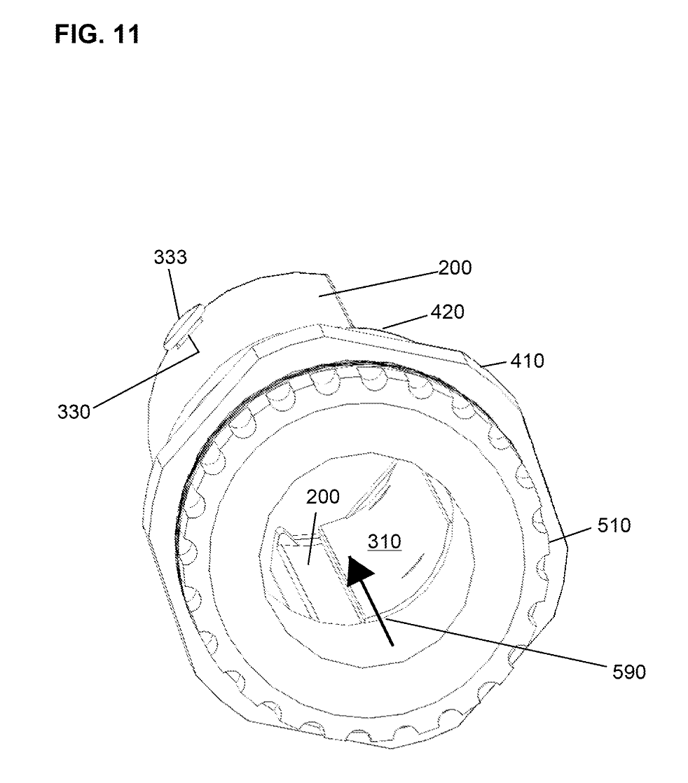

FIG. 11 shows the placement tower assembly 1500 illustrated in FIG. 10 from approximately the top of the device. No additional elements are shown, but this view illustrates very clearly the blade pin inner lumen 590 which extends down through the entire placement tower assembly 1500.

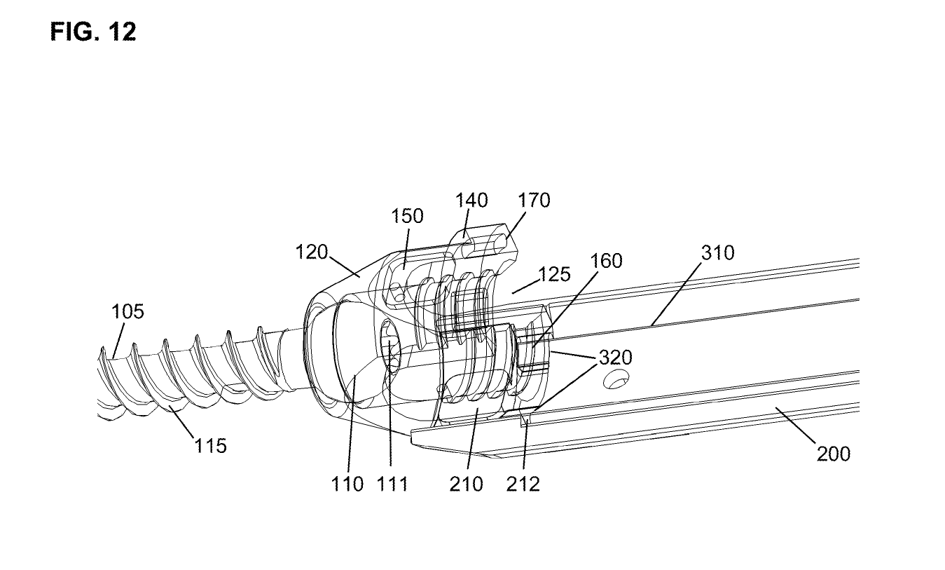

FIG. 12 illustrates a portion of the engagement of the bone screw and housing system 100 and one half of the placement tower assembly 1500 (for visualization purposes). No new or additional elements are shown. This figure is provided to demonstrate the connection between the placement tower assembly 1500 and the bone screw and housing system 100.

In operation, the outset housing clip 210 can fit or snap into the housing clip accepting inset 150 while the housing upper flange 140 first or snaps into the inset flange slot 212. The translating pegged blade 310 can be translated down (as disclosed above) to cause the at least one housing retaining peg 320 to translate down into the at least one peg accepting slot 170

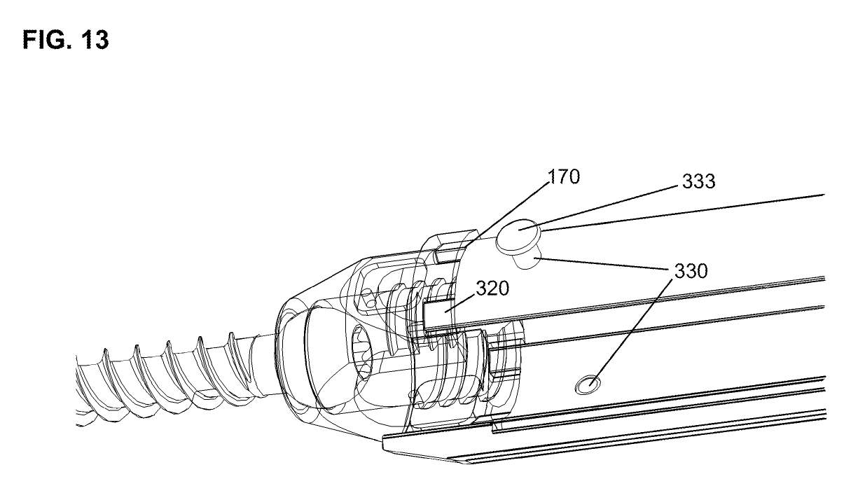

FIG. 13 shows the elements illustrated in FIG. 12 with the addition of a second translating pegged blade 310 and another retaining pin 330 and a retaining pin head 333. Note that the housing retaining blade 200 that would normally reside between the outer surface of the translating pegged blade 310 and the retaining pin head 333 is not shown for visualization purposes. As can be easily seen, when in an engaged configuration, the at least one housing retaining peg 320 resides in the at least one peg accepting slot 170.

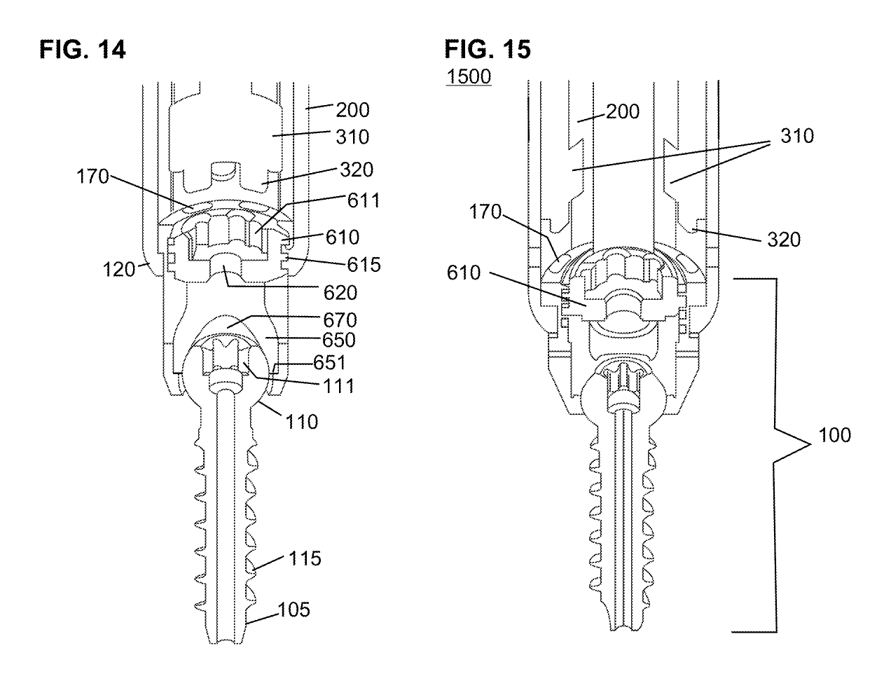

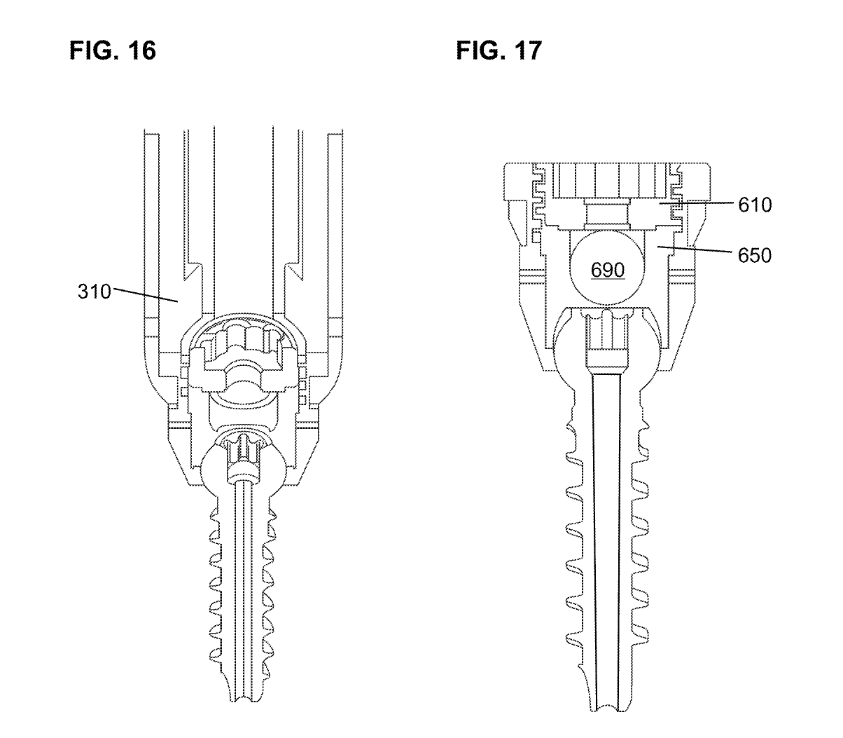

FIG. 14, FIG. 15, FIG. 16, and FIG. 17 show transected partial views of the placement tower assembly 1500 engaging the bone screw and housing system 100. FIG. 16 is an enlarged view of FIG. 15 without as many of the element lead lines to allow less unobstructed view of the assembly. The bone screw and housing system 100 is the same as has been disclosed above as is the placement tower assembly 1500. In addition to those elements disclosed above, FIGS. 14-17 include a locking cap 610 which has a locking cap central lumen 620, locking cap threads 615, and locking cap driving inset portion 611. FIGS. 14-17 also show a bone screw crown 650 which includes a bone screw crown central lumen 670, a bone screw crown lower flange 651 and a bone screw crown rod trough 675. The proximal face of the locking cap 610 may have one or more markings to help align components during use of the locking cap 610.

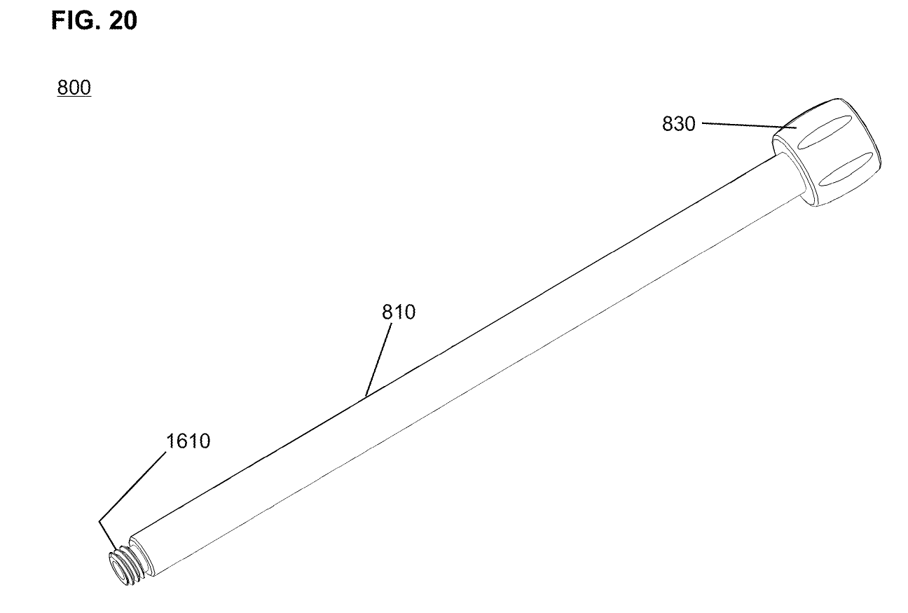

In operation, the bone screw 105 can be placed through the housing lower opening 130 of housing 120 then the bone screw crown lower flange 651 can be placed over the bone screw head 110. The placement tower assembly 1500 can be attached to the housing 120 to secure it and facilitate manipulation during insertion into the body. A fusing rod 690 (shown in FIG. 17) can be placed in the trough created by the bone screw crown rod trough 675. In further operation, the locking cap 610 can be screwed into the housing 120 via the housing internal threads 160. The thread type may be as shown in FIG. 20 for the threaded tip 1610 for the screw driving rod retainer 800 (discussed below) or could be some other thread type including flange threads. As the locking cap 610 is threaded in, it will eventually contact the upper surfaces of the fusing rod 690 and the bone screw crown 650. As the locking cap 610 is screwed down tightly, the locking cap 610 can cause force to be simultaneously applied to the fusing rod 690 and the bone screw crown 650, causing the fusing rod 690 to be forced down onto the channel of the housing 120 and the bone screw crown 650 forced down onto the bone screw head 110. The force of the fusing rod 690 pushing against the channel of the housing 120 can effectively secure the fusing rod 690 in the housing 120. In a similar manner, the bone screw crown 650 forces the bone screw head 110 against the lower surfaces of the housing 120 to fix the position of the bone screw head 110 against the housing 120. In this manner, the rod 690 and the bone screw head 110 can be put into a secure and fixed position.

In some examples, the locking cap 610 includes a locking cap central lumen 620.

In some examples, the bone screw crown rod trough 675 is the same size as the “U” created by the housing 120 such that the surfaces are substantially flush with each other. In these examples, when installed, the fusing rod 690 will have substantially equal and complete contact with the bottom of the “U” created by the bone screw crown rod trough 675 and the housing 120. In another example, the bone screw crown rod trough 675 is slightly larger than the “U” created by the housing 120 such that when the fusing rod 690 is installed, it contacts substantially only the “U” created by the housing 120 and not the bone screw crown rod trough 675. In still another example, the bone screw crown rod trough 675 is slightly smaller than the “U” created by the housing 120 such that when the fusing rod 690 is installed, it contacts substantially only the bone screw crown rod trough 675 and not the “U” created by the housing 120.

In some examples, the bone screw crown central lumen 670 which is aligned vertically along the central longitudinal axis of the housing 120 is just large enough to fit a driving device down through its lumen to reach the bone screw driving inset portion 111 and drive the bone screw 105. In such examples, the bone screw 105 can be driven into bone while in the housing while the bone screw crown 650 is in place. In other examples, there is no bone screw crown central lumen 670. In these examples, the bone screw 105 can be driven into the bone while in the housing 120, but the bone screw crown central lumen 670 cannot be present until after the bone screw 105 is set as desired.

In some examples, the bone screw crown lower flange 651 of the bone screw crown 650 extends down into the housing 120 and substantially fills the space surrounding the bone screw head 110. In other examples, the bone screw crown lower flange 651 of the bone screw crown 650 extends down into the housing 120 only partially and therefore is in contact with only a portion of the upper crown of the bone screw head 110.

In some examples, the locking cap driving inset portion 611 is adapted to be threaded into the housing internal threads 160 of the housing 120 by using a driving instrument.

FIG. 14, FIG. 15, FIG. 16, and FIG. 17 illustrate the locking cap 610 threaded fully into the housing internal threads 160 of the housing 120 and pushing down on the bone screw crown 650 such that the bone screw crown 650 and bone screw crown lower flange 651 of the bone screw crown 650 are in contact with the bone screw head 110. FIG. 14 and FIG. 15 illustrate the placement tower assembly 1500 holding the bone screw and housing system 100 wherein the translating pegged blades 310 are disengaged and the housing retaining pegs 320 are disengaged from the peg accepting slots 170.

By contrast, FIG. 16 illustrates the same view as FIG. 15 except that the translating pegged blades 310 of the placement tower assembly 1500 are both engaged and therefore the housing retaining pegs 320 of the translating pegged blades 310 are engaged with the peg accepting slots 170 of the housing 120 thereby promoting temporary retention of the housing 120 of the bone screw and housing system 100 by the placement tower assembly 1500. FIG. 14, FIG. 15, and FIG. 16 are shown without the fusing rod in an effort to reveal the interior relationship of components. By contrast, FIG. 17 illustrates the fusing rod 690 in place within the bone screw crown 650 on top of the bone screw crown 650 and below the locking cap 610.

In some examples, the bone screw crown 650 is configured such that when the fusing rod 690 is in place in the “U” and the bone screw crown rod trough 675 contacts only the bone screw crown 650 on its lower side. In these examples, the locking cap 610 can push down simultaneously on the bone screw crown 650 and the fusing rod 690 causing the fusing rod 690 to also push down onto the channel of the housing 120. The bone screw crown 650 therefore is the member which directly touches the bone screw head 110 and directly applies pressure to the bone screw head 110 to fix its position within the housing.

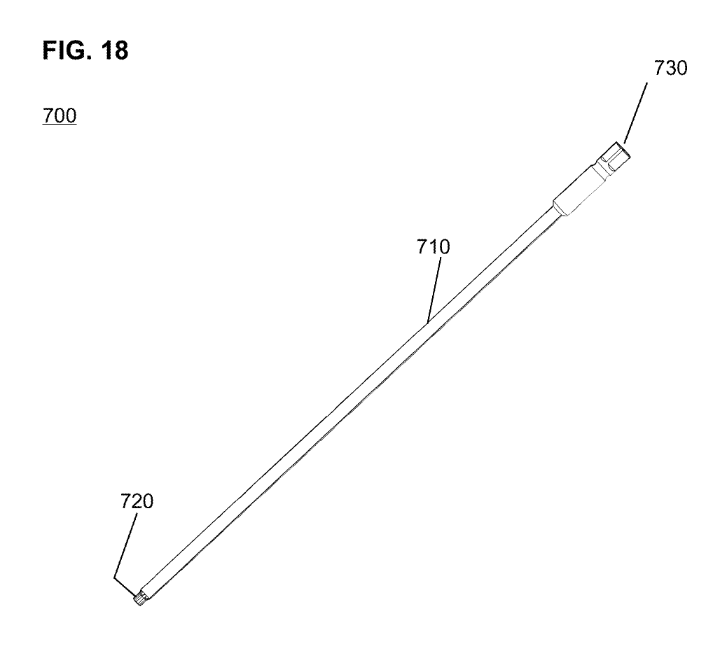

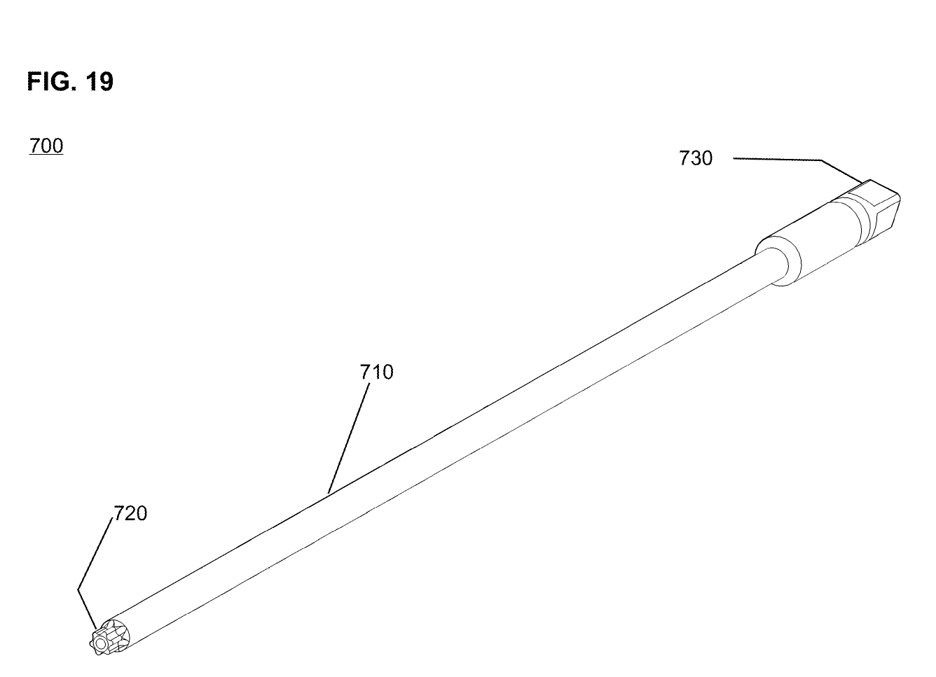

FIG. 18 and FIG. 19 illustrate a bone screw driving rod 700. The bone screw driving rod 700 includes a bone screw driving rod shaft 710 a bone screw driving rod head 720 and a bone screw driving handle adapter 730. In operation, the bone screw driving rod 700 is inserted down through the blade pin inner lumen 590 of the placement tower assembly 1500 to reach the bone screw driving inset portion 111 of the bone screw head 110 of the bone screw 105. In further operation, the bone screw driving rod head 720 of the bone screw driving rod 700 mates with the bone screw driving inset portion 111 of the bone screw head 110 and can be turned either clockwise or counterclockwise using a handle (not shown) attached to the bone screw driving handle adapter 730 to insert or remove the bone screw 105 as desired.

FIG. 19 illustrates the bone screw driving rod 700 from a perspective view showing the bone screw driving rod head 720 in more detail. Those of skill in the art will recognize that other bone screw driving rod heads may be used with correspondingly shaped bone screw heads.

In some examples, the bone screw driving rod shaft 710 of the bone screw driving rod 700 is at least marginally longer than the axial length of the placement tower assembly 1500.

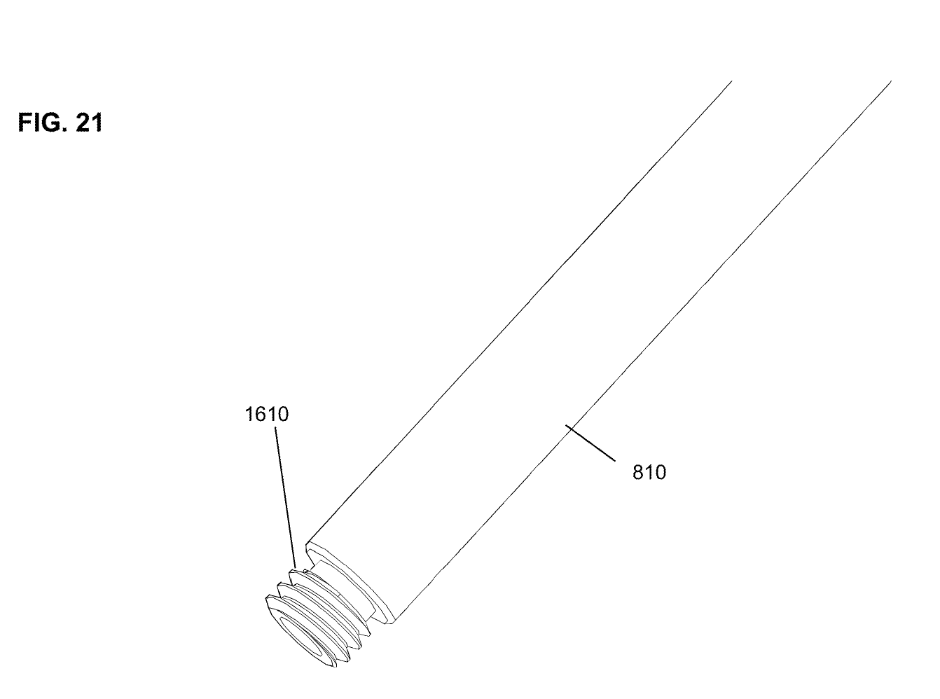

FIG. 20 and FIG. 21 a screw driving rod retainer 800 with a rod shaft 810 and a threaded tip 1610, and a retainer rod head 830. Note that the threaded tip 1610 is sized with the male threads of the locking cap 610 so that the screw driving rod retainer 800 may engage the housing internal thread 160. In operation, the screw driving rod retainer 800 is inserted down through the blade pin inner lumen 590 of the placement tower assembly 1500 to reach the housing internal threads 160 of the housing 120. In further operation, threaded tip 1610 which is disposed on the end of the rod shaft 810 can be turned either clockwise or counterclockwise using the retainer rod head 830 to engage or disengage the threaded tip 1610 from the housing 120.

FIG. 21 illustrates the screw driving rod retainer 800 from a perspective view showing the threaded tip 1610 in more detail. The center of the screw driving rod retainer 800 is hollow/cannulated to enable the bone screw driving rod shaft 710 to slide through.

The combination of bone screw driving rod 700 and screw driving rod retainer 800 form bone screw driving assembly 780. Bone screw driving assembly 780 can be advanced through the center of the placement tower assembly 1500 to allow the bone screw driving rod head 720 to engage the bone screw driving inset portion 111. As the bone screw driving assembly 780 allows relative rotational and translational motion between the screw driving rod retainer 800 and the bone screw driving rod 700, the screw driving rod retainer 800 may be rotated to threadedly engage the threaded tip 1610 with the housing internal threads 160.

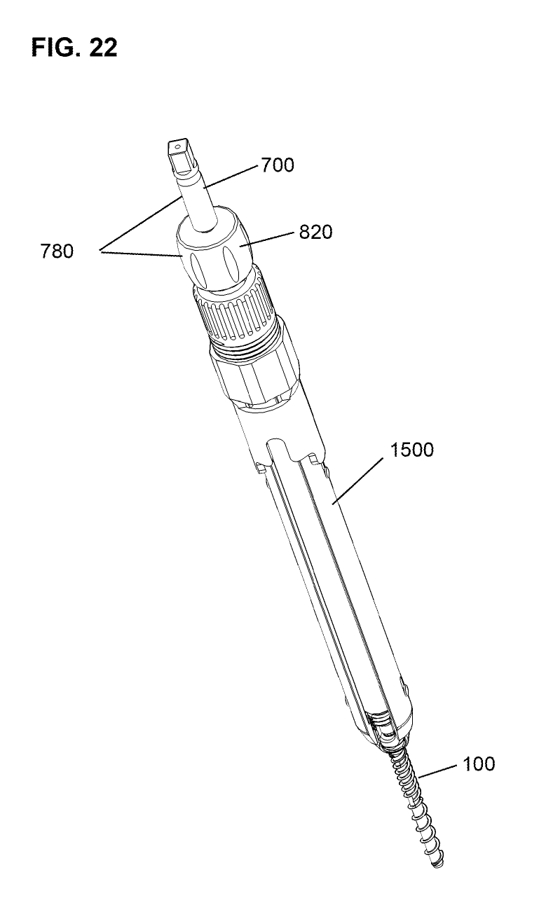

FIG. 22 illustrates the placement tower assembly 1500 holding the bone screw and housing system 100 as discussed above with the bone screw driving assembly 780 in place. In this configuration, the bone screw driving rod 700 can be turned to drive the bone screw 105 into bone as desired, and then the bone screw driving assembly 780 can be removed.

After removal of bone screw driving assembly 780, the placement tower assembly 1500 can be rotated or swiveled to align the “U” of the housing 120 and the bone screw crown rod trough 675 with the direction needed for the fusing rod 690. Once in the correct confirmation, the fusing rod 690 can be inserted into the “U” and the bone screw crown rod trough 675 then the locking cap 610 may be engaged with the housing internal threads 160 and locked down. The locking cap 610 can be inserted tightly, thereby clamping down on the bone screw crown 650 and fusing rod 690 causing the housing 120 and fusing rod 690 to be fixed with respect to the bone screw 105 as discussed with respect to FIGS. 12-17.

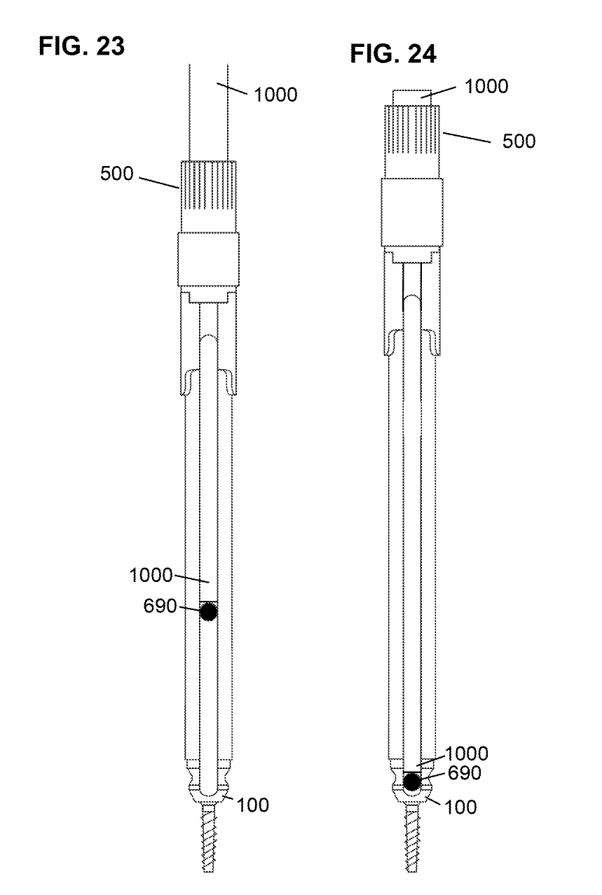

FIG. 23 and FIG. 24 illustrate the optional use of a persuader 1000. In operation, the placement tower assembly 1500 is attached to the bone screw and housing system 100 via the housing 120 and the bone screw 105 is inserted into bone as disclosed above. After the bone screw driving assembly 780 is removed from the placement tower assembly 1500 prior to the insertion of the locking cap 610 into the housing 120 the blade pin inner lumen 590 is open. A persuader 1000 can then be inserted down the open blade pin inner lumen 590 of the placement tower assembly 1500 to tamp or persuade the fusing rod 690 to seat fully into the bottom of the “U” of the housing 120 and the bone screw crown rod trough 675. Thus, as the persuader 1000 moves down towards the housing 120, the fusing rod 690 is moved into position.

Downward force conveyed through the persuader 1000 to the fusing rod 690 may be from a hand pushing on the proximal end of the persuader 1000. Downward force conveyed through the persuader 1000 to the fusing rod 690 may be from a mallet or slap hammer hand tapping on the proximal end of the persuader 1000. Downward force conveyed through the persuader 1000 to the fusing rod 690 may be from a threaded engagement of the persuader 1000 with threads inside the placement tower assembly 1500 (threads not shown).

FIG. 24 illustrates the persuader 1000 tamping or persuading the fusing rod 690 to seat fully. After the fusing rod 690 has seated fully, the persuader 1000 can be removed and a locking cap driver (not shown) may be inserted to lock the assembly with the locking cap 610.

Alternatively, the persuader 1000 may be cannulated so that the locking cap driver with engaged locking cap 610 may be inserted through the persuader 1000 so that the persuader 1000 may be used to help maintain the position of the fusing rod 690 as the locking cap 610 is engaged with the housing internal threads 160 and makes contact with the fusing rod 690.

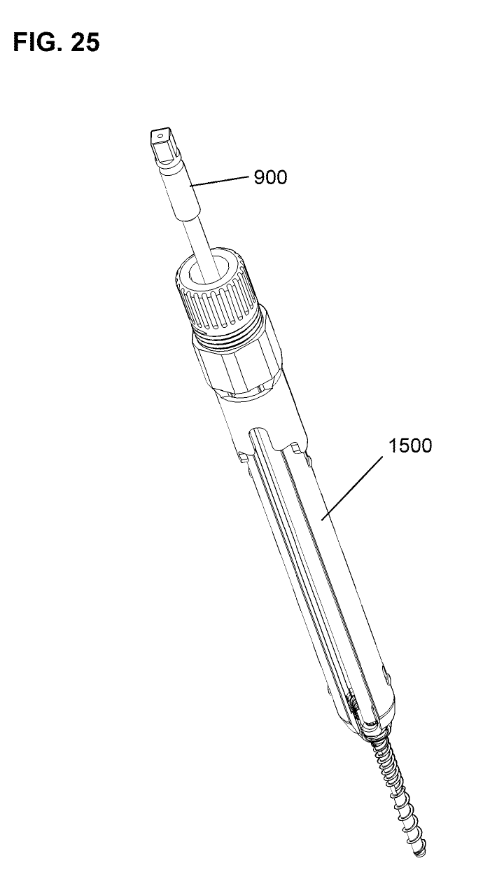

FIG. 25 shows a locking cap driver 900 that may be inserted through the placement tower assembly to tighten the locking cap 610 (not shown here). The locking cap 610 may be connected to a distal tip of the locking cap driver 900 using any conventional technique, including a press fit or a spring retaining hex. This locking cap driver 900 may be used as shown or through the cannulated persuader 1000. A torque wrench (not shown) set to only apply torque below a set point may be used to rotate the locking cap driver 900 so as to avoid over tightening the locking cap 610. A lockdown torque will be achieved when a torque wrench with a torque limiting handle limited to provide a maximum torque of between about 80 and about 110 in-lbs.

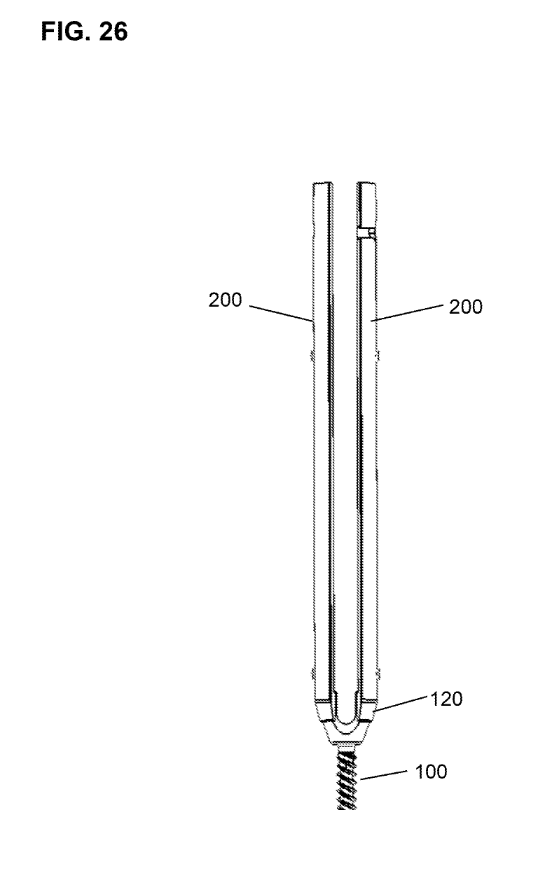

FIG. 26 illustrates the placement tower assembly 1500 attached to the bone screw and housing system 100 via the housing 120 with select components removed. In operation, during surgery the surgeon can easily remove both the locking collar 400 and blade pin 500 to improve visualization, or to facilitate a bailout. For example, should become difficult to see the housing 120 or other components below the incision site, the surgeon can remove all components of the placement tower assembly 1500 aside from the twin housing retaining blades 200 (and the affixed members, namely the translating pegged blade 310) thereby allowing the surgeon to pry the twin housing retaining blades 200 apart to allow improve visualization. Once a surgeon has decided on a path of action, the twin housing retaining blades 200 can be returned to their parallel, cylindrical confirmation (still holding the housing 120) and the locking collar 400 and blade pin 500 can be returned thereby once again fixing the twin housing retaining blades 200 to the housing 120 allowing the surgeon to continue the surgery.

The pedicle screw and rod based spinal fusion systems disclosed herein provide for an improved surgical efficacy because they allow for improved attachment between the placement tower assembly 1500 and the bone screw and housing system bone screw and housing system 100, improve visualization during surgery, and promote decisional flexibility and the ease of bailout during surgery. The systems disclosed do not require the removal of the entire system to improve visualization—rather, they allow for easy disassembly and subsequent reassembly during surgery to facilitate visualization, and flexibility of working space.

Of course, the foregoing description is of certain features, aspects and advantages of the present invention, to which various changes and modifications can be made without departing from the spirit and scope of the present invention. Thus, for example, those of skill in the art will recognize that the invention can be embodied or carried out in a manner that achieves or optimizes one advantage or a group of advantages as taught herein without necessarily achieving other objects or advantages as can be taught or suggested herein. In addition, while a number of variations of the teachings of this disclosure have been shown and described in detail, other modifications and methods of use, which are within the scope of this invention, will be readily apparent to those of skill in the art based upon this disclosure. It is contemplated that various combinations or sub-combinations of the specific features and aspects between and among the different examples can be made and still fall within the scope of the invention. Accordingly, it should be understood that various features and aspects of the disclosed examples can be combined with or substituted for one another in order to form varying modes of the discussed devices, systems and methods (e.g., by excluding features or steps from certain examples, or adding features or steps from one example of a system or method to another example of a system or method).

Provision of Therapy.

After creating access to the targeted posterior spinal vertebral levels, and aligning and stabilizing/fixing them using the methods as disclosed herein, additional therapy may be provided. One form of therapy is to fuse the selected spinal levels together. Spinal fusion typically involves the use of osteogenic, osteoconductive, or osteoinductive material (bone graft). One process to promote fusion is to insert quantities of one or more fusion promoting materials into the areas to be fused. Bone graft is the material that is used to promote bone growth and forms the scaffold that bridges the adjacent vertebral bodies comprising a motion segment in the spine. The fused portions of the vertebrae do not move with respect to one another. It is useful to have one name for the variety of materials used to promote fusion. Thus, fusion promoting materials including osteogenic, osteoconductive, and/or osteoinductive material are collectively described herein as bone graft, whether the material is autograft or allograft and various bone graft substitutes or bone graft extenders. Various techniques for promoting effective fusion of adjacent vertebrae are well known to those of skill in the art so a minimal summary is sufficient for this document. The pedicle screw systems of the present disclosure may be used in conjunction with bone graft types that are autologous or allogenic, e.g., grafts from the iliac crest, rib, or tibia/fibula donor sites. Autograft, a combination of autograft and allograft, or allograft alone may be used. As those of skill in the art will be familiar, for example, bone graft may be delivered via facet fusion through the same incision made for insertion and deployment of the fixation systems as presently disclosed or by means of a new incision created over a facet. A burring tool may be used on the facet joint and bone graft placed around the prepared facet. With pedicles, fusion is also possible by means of open or posterior lateral procedures.

Example Method of Deployment.

While surgeons may alter the sequence of deployment to meet with particular needs of a patient procedure or the personal preferences of the surgeon, the sequence for most surgeons, most of the time will be as described below. Frequently, more than one fusing rod 690 is used in a surgical procedure. The deployment steps set forth below may be repeated, done in parallel, or some combination of thereof to deploy one or more additional fusing rods 690.

Those of ordinary skill in the art will recognize that a process to deploy an implant of any type is frequently assisted by fluoroscopic imaging to assess placement of guidewires and components during the process. Anterior/Posterior imaging is particularly useful for this procedure as is proper placement of a patient to provide access to the pedicles.

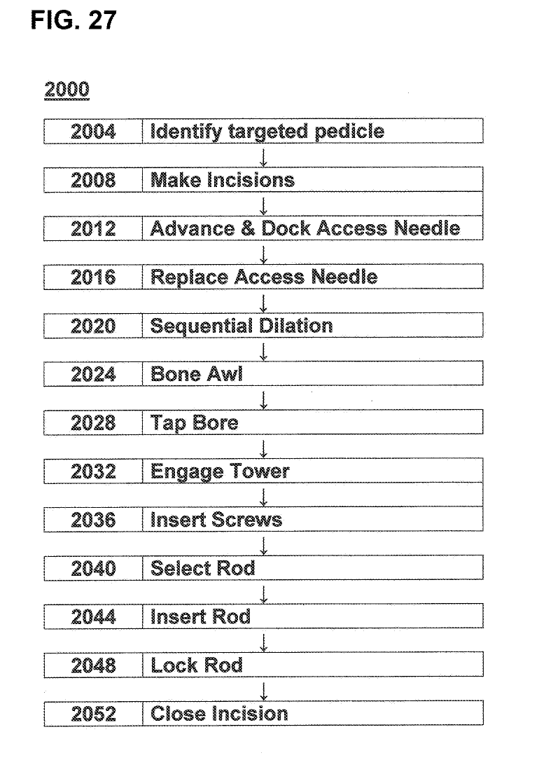

FIG. 27 is a flow chart for the sequence of deployment 2000.

Step 2004—Identify Targeted Pedicle.

In one example of deployment of the present pedicle fixation system, prior to making an incision, the surgeon first identifies a lateral border(s) of the pedicles to be targeted using a metallic object, such as a guidewire, in conjunction with anterior/posterior (A/P) fluoroscopy, and draws a line on the patient's skin along the lateral borders. The superior borders of the pedicles are then identified at each level in the same manner and by drawing lines identifying these borders.

Step 2008—Make Incisions.

Incisions are made between about 2 cm and about 3 cm lateral to the first lines drawn (i.e., identifying the lateral borders of the pedicles), caudal to the superior borders, in one of two exemplary manners noted below, depending on the type of rod placement selected. If using a percutaneous rod insertion tool, incisions of about 1 cm long are made at each level targeted, caudally from the lines identifying the superior borders of the pedicle. If using a non-percutaneous “mini-open” rod insertion tool, one, slightly longer incision is made between the lines identifying the superior borders of the most cephalad and most caudal levels. In this method as just described, the tissue initially may be “finger dissected” which assists with subsequent rod delivery.

Step 2012—Advance & Dock Access Needle.

Using an access needle, the pedicles at levels where screws are being placed are targeted using A/P and lateral fluoroscopy. This is done by placing the tip of the access needle into the incision and advancing the needle to the junction of the transverse process and the facet joint, in line with the pedicle by tapping it with a mallet. The needle is advanced approximately a third of the way into a vertebral body.

Step 2016—Replace Access Needle.

Once the Access Needle is docked in the vertebral body, an inner stylet comprising an aggressive distal tip is used to puncture the pedicle and it is then removed and a K-wire or guidewire is inserted to approximately ¾depth in the vertebral body.

Step 2020—Sequential Dilation.

Next, using a sequential (circumferential) dilation procedure, the incision is dilated down to the bone. In one example, at least one dilator in the sequence is made of Radel®, a radiolucent and non-conductive polymer (preferable when using neuromonitoring).

Step 2024—Bone Awl.

Following removal of the initial dilators a bone awl may be inserted over a guidewire (or K-wire) to breach through the cortical bone. In a preferred example, there is an approximate 10 mm stop on the bone awl.

Step 2028—Tap Bore.

Next, a tap marked with depth indicators is used (being careful not to tap past the end of the guidewire) to appropriately size the screw to be selected. The outer diameters of taps are 1:1 with the screw diameters while the minor diameter of the tap is slightly smaller than the minor diameter of the screw. A neuromonitoring probe may be used to check the position of the tap.

Step 2032—Engage Tower.

The bone screw 105 fits or slips down through the housing lower opening 130 in the housing 120. Engage housing 120 with the placement tower assembly 1500 (See FIG. 12). Laser markings may be used to help with alignment.

Step 2036—Insert Screws.

Insert the distal end of the bone screw driving assembly through the placement tower assembly. Seat the distal end of the screwdriver in the driver engagement section of the bone screw. Rotate the bone screw driving assembly to engage the distal tip of the screw driving rod retainer with the housing internal threads. Drive the bone screw into the bone. Remove the K-Wire. Repeat with other bone screws. When all screws are inserted, adjustments may be made in order to align the screws. Optionally, the adjustment to screw height may be made using an ancillary driver—not shown but similar to locking cap driver 900 in that the ancillary driver does not have a corresponding screw driving rod retainer 800.

Step 2040—Select Rod.

Next, a rod measuring tool is used to measure for the rod length for the fusing rod 690, by seating the ends of the tool fully into the screw heads, and then taking a reading to select the appropriate rod length, while accounting for overhang and/or distraction in length calculations. When using a percutaneous rod insertion tool, a bulleted rod (not shown) is selected, whereas a “mini-open” rod insertion tool is used in conjunction with a standard or bulleted rod. Both straight and pre-lordosed rods may be used with the presently disclosed system.

Step 2044—Insert Rod.

A fusing rod 690 is attached to the rod inserter and inserted into the screw heads. When using a percutaneous rod insertion tool, the fusing rod 690 is inserted along one of the outer screw's towers, through a slot in a tab. As the fusing rod 690 nears the tulip head of the screw, the rod insertion tool should be angled or articulated so that the tip of the fusing rod 690 crosses through the next screw head, and so on, depending on the number of levels, until the fusing rod 690 is through the slots of all screws on that side. When using a “mini-open” rod insertion tool is, a blunt dissector is used to clear tissue from a channel between towers. The rod insertion tool is situated between the screw heads so that the rod will seat fully. Seating of the rod into the screw heads may be further facilitated by use of a tower reduction system or a rod pusher tool.

For example, to more effectively place the fusing rod 690, the bone screw driving assembly 780 can be removed from the placement tower assembly 1500 prior to the insertion of the locking cap 610 into the housing 120 thereby leaving a completely open blade pin inner lumen 590. A persuader 1000 can then be inserted down the open blade pin inner lumen 590 of the placement tower assembly 1500 to tamp or persuade the fusing rod 690 to seat fully into the bottom of the “U” of the housing 120 and the bone screw crown rod trough 675.

Step 2048—Lock Rod.

Insert the distal end of the locking cap driver 900 for the locking cap 610 with an engaged locking cap 610 through the placement tower assembly 1500 to engage housing internal threads 160 of the housing 120. Connect a wrench to the placement tower assembly 1500 to preclude rotation of the placement tower assembly 1500 while applying torque to tighten the locking cap 610. A lockdown torque will be achieved with a torque limiting handle set to limit the application of torque to between about 80 and about 110 in-lbs.

Next, the rod insertion tool (not shown) and the locking cap driver 900 are removed.

Note—If compression or distraction are needed a compressor/distractor tool is attached to the placement tower assemblies 1500 and the appropriate force is applied to impose the desired movement of the vertebrae. While compression/distraction is being applied, the fusing rod 690 is locked in place by using a torque limiting tool, e.g., a torque limiting T-handle. The compressor/distractor tool is then removed, and following final adjustments, any remaining locking caps are locked with a torque limiting handle.

Step 2052—Close Incisions.

Following subsequent removal of the placement tower assemblies 1500 and all other instrumentation, the incision is closed.

Bone Screws

During the discussion of FIG. 1, the bone screw 105 was introduced. While it was noted that the bone screw may have one or more sets of bone screw threads, the focus of that part of the disclosure was the polyaxial aspects of the bone screw and housing system 100 and the interaction with the placement tower assembly 1500 and other tools used during deployment. This portion of the disclosure turns the focus onto the bone screw 105 and the bone screw threads 115.

To minimize the risk of confusion, the discussion of several disclosed bone screws which may be used in a manner described above, will use non-overlapping element numbers with the discussion above.

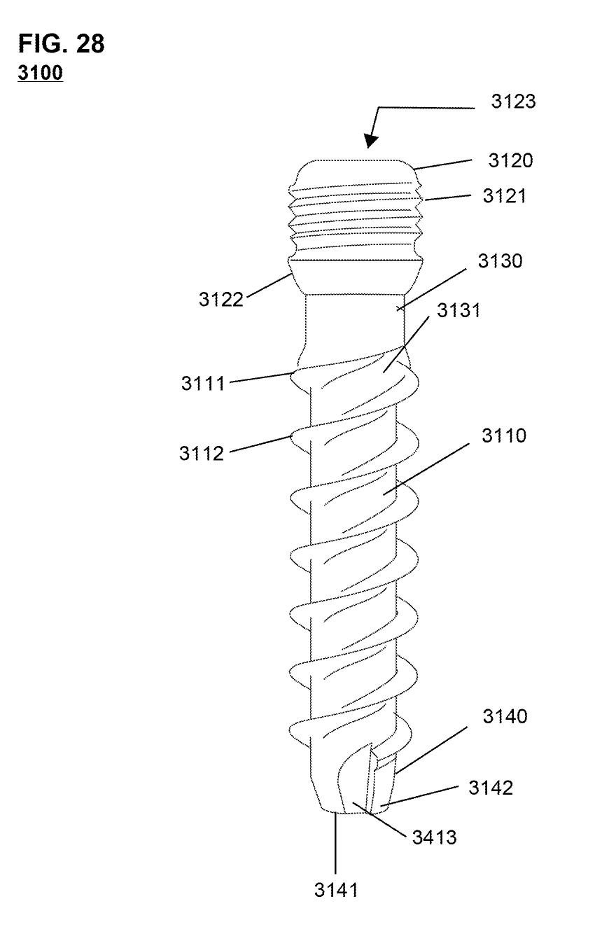

FIG. 28 illustrates a side view of one example of a pedicle screw 3100. In this illustrated example, the pedicle screw 3100 can include a shank 3110. In some examples the shank 3110 is an elongate member extending along a screw axis. The shank 3110 can include a proximal portion 3130 and a distal portion 3140. The pedicle screw 3100 can include a head 3120, attached to the proximal portion 3130 of the shank 3110. A distal tip 3141 can be located at the end of the distal portion 3140 furthest from the head 3120.

As shown in FIG. 28, the pedicle screw 3100 can include a male first thread 3111 located on the external surface of the shank 3110 extending from the proximal portion 3130 to the distal portion 3140.

With continued reference to FIG. 28, the pedicle screw 3100 can include a male second thread 3112 located on the external surface of the shank 3110 extending from the proximal portion 3130 to the distal portion 3140.

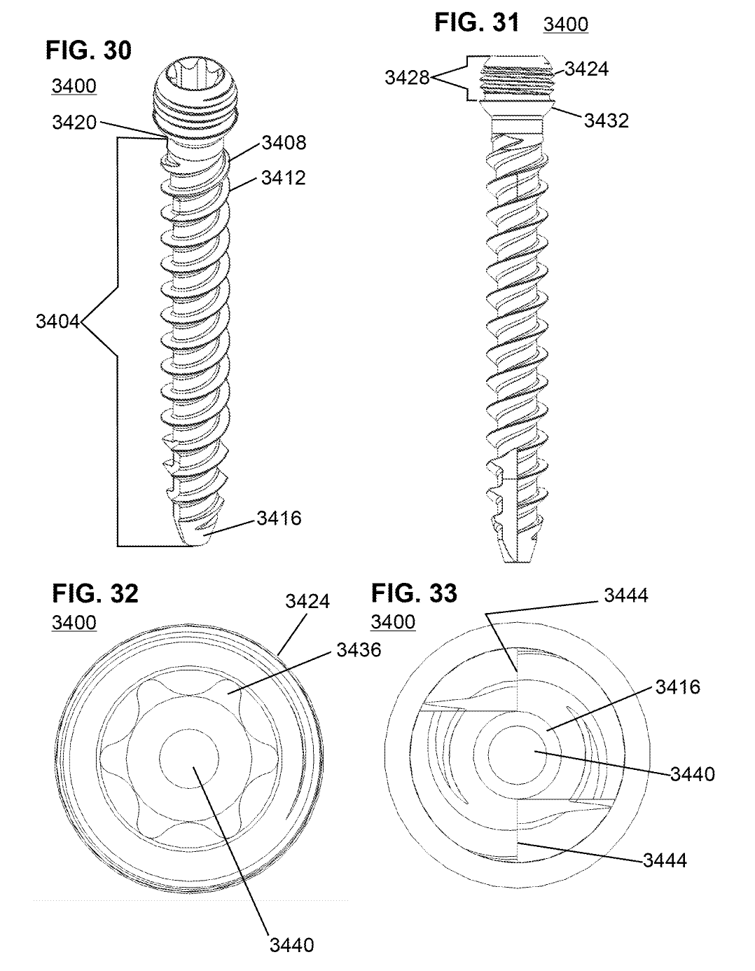

As shown in FIG. 30, the second thread 3112 can be offset approximately 180 degrees from the first thread 3111. The first thread 3111 and second thread 3112 can each have a constant pitch and the spacing between the first thread 3111 and second thread 3112 along the length of the shank 3110 can be constant. In the illustrated arrangement, the first thread 3111 and second thread 3112 can have a substantially constant major diameter. However, in other arrangements, the major diameter of the first thread 3111 and second thread 3112 can vary depending on the application of the pedicle screw 3100.

As shown in FIG. 28, the major diameter of the first thread 3111 and second thread 3112 can decrease near the distal tip 3141 of the pedicle screw 3100. In FIG. 28, the first thread 3111 and second thread 3112 can also have substantially constant minor diameter. However, as shown in FIG. 28 and described below, the distal end of the shank 3110 can taper inwardly and the proximal end of the shank 3110 can expand to a larger diameter. The first thread 3111 and second thread 3112 of the illustrated example are continuous and uninterrupted along their length. As mentioned previously, the pedicle screw 3100 can include a distal taper 3142 to aid in insertion of the pedicle screw 3100 into a vertebra. The pedicle screw 3100 can also include a proximal taper 3131 near the proximal initiation points of the first thread 3111 and second thread 3112 to prevent an abrupt end to the threads and provide a gradually increasing resistance during insertion to deter advancing the pedicle screw 3100 more deeply than intended.

The first thread 3111 and second thread 3112 can be configured such that rotation of the pedicle screw 3100 in a first direction advances the pedicle screw 3100 into bone. As shown in FIG. 28, the head 3120 can include a head thread 3121 on its outer surface. In some examples, the head thread 3121 is configured to accept a female threaded collar (not illustrated), which when engaged with the head thread 3121 can form part of a head of the pedicle screw 3100.

In some examples, the head thread 3121 can be configured such that rotation of a collar in a second direction advances the collar towards the distal tip 3141 of the pedicle screw 3100 and couples the collar to the head 3120.

In some examples, the first thread 3111 and second thread 3112 can be a right hand thread and the head thread 3121 can be a left hand thread, or vice versa. In other examples, the first thread 3111, second thread 3112, and head thread 3121 may all share the same style thread, be it right handed or left handed.

In some examples, as illustrated in FIG. 28, the proximal starts for the first thread 3111 and the second thread 3112 can be offset substantially the same distance from the head 3120. In some examples, the distal ends of the first thread 3111 and the second thread 3112 can be offset substantially the same distance from the head 3120.

The head 3120 can have a larger diameter than the shank 3110 as shown in FIG. 28. In some examples, the distal part of the head 3120 may include a receiver mating surface 3122 configured to complement the collar and interact with the housing 120 introduced in FIG. 1. In some examples, the head 3120 may include a tool receiving feature 3123 (not visible illustrated), such as a hex fitting, hexalobe fitting, or any of the many driver engagement shapes used to drive screws. The tool receiving feature 3123 is configured to accept a corresponding tool tip and allow the tool to transfer a torque to the pedicle screw 3100. In some examples, the pedicle screw 3100 can include a tapping feature 3143 constructed to remove material and aid in the insertion of the pedicle screw 3100 into bone. In some examples, the pedicle screw 3100 may be cannulated to allow delivery over a guidewire.

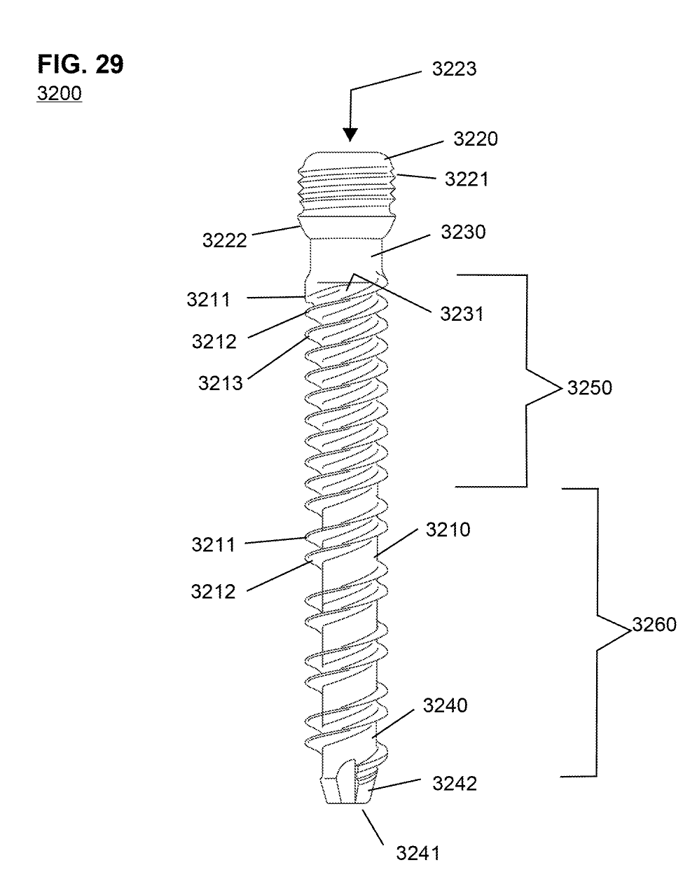

FIG. 29 illustrates a side view of another example of a pedicle screw 3200, which includes a shank 3210 that forms an elongate member extending along a screw axis. The shank 3210 can include a proximal portion 3230 and a distal portion 3240. The pedicle screw 3200 can include a head 3220, attached to the proximal portion 3230 of the shank 3210. The pedicle screw 3200 can also include a distal tip 3241 located at the end of the distal portion 3240 furthest from the head 3220.

As shown in FIG. 29, the pedicle screw 3200 can include a male first thread 3211 located on the external surface of the shank 3210 extending from the proximal portion 3230 to the distal portion 3240.

The pedicle screw 3200 can also include a male second thread 3212 located on the external surface of the shank 3210 extending from the proximal portion 3230 to the distal portion 3240.

In some examples, the pedicle screw 3200 can include a male third thread 3213 located on the external surface of the shank 3210 extending from the proximal portion 3230 towards the distal portion 3240.

The second thread 3212 can be offset approximately 120 degrees from the first thread 3211 and the second thread 3212 can be offset approximately 3120 degrees from the third thread 213. The third thread 213, in turn, can be offset approximately 120 degrees from the first thread 3211. In some examples, the first thread 3211, second thread 3212, and third thread 3213 can have constant pitch. In some examples, the spacing between the first thread 3211, second thread 3212, and third thread 3213 along the length of the shank 3210 is constant. In the example of FIG. 29, the first thread 3211, second thread 3212, and third thread 3213 can have substantially constant major diameter. In other arrangements, the major diameter of one or more of the first thread 3211, second thread 3212, and third thread 3213 can vary depending on the application of the bone screw. As shown in FIG. 29, the major diameter of the first thread 3211 and second thread 312 can decrease at a distal taper 3242 near the distal tip 3241 of the pedicle screw 3200. The first thread 3211, second thread 3212, and third thread 3213 can have substantially constant minor diameter. In some examples, the first thread 3211, second thread 3212, and third thread 3213 are continuous and uninterrupted along their length.

As mentioned above, the pedicle screw 3200 can include a distal taper 3242 to aid in insertion of the pedicle screw 3200 into a vertebra. The pedicle screw 3200 can also include a proximal taper 3231 near the proximal starts of the of the first thread 3211, second thread 3212, and third thread 3213 to prevent an abrupt proximal end to the threads and provide a gradually increasing resistance during insertion to deter advancing the pedicle screw 3200 more deeply than intended.

In some examples, the first thread 3211, second thread 3212, and third thread 3213 can be configured such that rotation of the pedicle screw 3200 in a first direction advances the pedicle screw 3200 into bone. In some examples, the head 3220 includes a head thread 3221 on its outer surface. In some examples, the head thread 3221 is configured to accept a female threaded collar (not shown here). As noted with respect to FIG. 28, the collar can form part of the head of the pedicle screw 3200 which can be received in the housing 120 (FIG. 1). Alternatively, the head may be without threading to receive a collar and may be used as is.

In some examples, the head thread 3221 can be configured such that rotation of a collar in a second direction advances the collar towards the shank 3210 and couples the collar to the head 3220. In other words, the first thread 3211, second thread 3212, and third thread 3213 can be a right hand thread and the head thread 3221 can be a left hand thread, or vice versa (not illustrated).

In other examples, the first thread 3211, second thread 3212, third thread 3213, and head thread 3221 may all share the same style thread, be it right handed or left handed.

As illustrated in FIG. 29, the proximal starts for the first thread 3211, the second thread 3212*, and the third thread 3213 can be offset substantially the same distance from the head 3220. As illustrated in FIG. 29, the distal terminations of the first thread 3211 and the second thread 3212 can be offset substantially the same distance from the head 3220. In the arrangement of FIG. 29, the distal termination of the first thread 3211 and the second thread 3212 can be offset a first distance from the head 3220 and the distal termination of the third thread 3213 can be offset a second distance from the head 3220, where the second distance is less than the first distance. In some examples, the second distance can be substantially shorter than the first distance. In some examples, the second distance can be between approximately 5 and 30 millimeters. In some examples, the second distance can be between approximately 15 and 25 millimeters. In some examples, the second distance can be approximately 20 millimeters. In some examples, this can result in an uneven spacing between threaded protrusions along at least a portion of the distal portion of the shank 3210 as illustrated in FIG. 29.

In some examples, the portion of the pedicle screw 3200 which includes at least a portion of the first thread 3211, second thread 3212, and third thread 3213 can be characterized as a first segment 3250. In some examples, the distances between the three thread protrusions along the length of the pedicle screw 3200 in the first segment 3250 is constant.

In some examples, the portion of the pedicle screw 3200 which is distal to the distal end of the third thread 3213 but before the distal terminations of the first thread 3211 and second thread 3212 can be characterized as a second segment 3260. In some examples, the distance between the thread protrusions along the length of the pedicle screw 3200 in the second segment 3260 is not constant, as illustrated in FIG. 29.

In some examples, the head 3220 can have a larger diameter than the shank 3210. In some examples, the distal part of the head may include a receiver mating surface 3222 configured to complement the collar and interact with other portions of the spinal fixation system (housing 120 from FIG. 1). As noted with respect to FIG. 28, the head 3220 may include a tool receiving feature 3223 (not illustrated) which is configured to accept a tool and allow the tool to transfer a torque to the pedicle screw 3200. In some examples, the pedicle screw 3200 can include a boring feature 3243 constructed to remove material and aid in the insertion of the pedicle screw 3200 into bone. In some examples, the pedicle screw 3200 may be cannulated to allow delivery over a guidewire.

FIG. 30 shows a top perspective view of bone screw 3400. Bone screw 3400 has a dual lead thread pattern on the screw shank 3404 with a first thread 3408 offset approximately 180 degrees from second thread 3412. The minor diameter of the screw shank 3404 is constant over the majority of the screw shank 3404 but tapers down at the distal end 3416 and flares out a the proximal end 3420 for the reasons previously discussed.

FIG. 31 is a side view of bone screw 3400. A third thread 3424 on the bone screw head 3428 may be used to secure a collar to the bone screw 3400. Use of a handedness for third thread 3424 that is the opposite of the handedness of the first thread 3408 and the second thread 3412 may reduce any likelihood of the collar to loosen while the bone screw 3400 is being advanced in the bone. A receiver mating surface 3432 may work to extend the curved surface of the collar to allow for polyaxial movement of the bone screw 3400 with collar within a housing 120 (FIG. 1).

FIG. 32 shows a top view of bone screw 3400. The start of the third thread 3424 is visible in FIG. 32. Also visible is a driver engagement section 3436. The driver engagement section may be a hex lobe, torx socket, or other driver pattern known in the art. The bone screw 3400 is cannulated so that the bone screw may be delivered over a guidewire. Cannula 3440 is visible in top view FIG. 32 and bottom view FIG. 33.

FIG. 31 shows boring feature 3444 constructed to remove material and aid in the insertion of the screw into bone. FIG. 33 shows that there is a pair of boring features 3444.

Component Details.

Materials Choices.

Choices for material for use in the various components comprised in the constructs and bone screws shown herein are machinable and medical grade, and include but are not limited to titanium or titanium alloys, cobalt-chromium alloys, and stainless steel alloys, or combinations thereof. Another material that may be used is Polyether ether ketone (PEEK) is a colorless organic polymer thermoplastic used in engineering applications.

These biocompatible materials can withstand sterilization techniques such as Ethylene oxide (EtO) gas, radiation, steam autoclaving, dry heat, and cold sterilization. Other desirable attributes are that the material is able to be imaged, e.g., visible via fluoroscopy, X-ray and/or computed tomography (CT); dimensionally stable, and with sufficient biomechanical properties (strength, stiffness, toughness) for intended use, e.g., is sufficiently stiff to allow a relatively thin wall. If needed, materials may be used with incorporated visualization markers, e.g. tantalum, although other materials may be used. The selected material(s) is preferably able to undergo surface treatments, such as bead blasting to promote anti-slippage, or coating such as with hydroxyapatite (HA) to promote bone in-growth.

Size.

The dimensions of the implants will be, in part, a function of the patient anatomy as well as the condition (e.g., depth, strength) of available bone. That is, dimensions (e.g., length, width, thickness) of the implants will be a function of the size of the patient as some patients have larger bones than other patients. Thus, the devices may be scaled to fit adults of smaller stature, e.g., the anterior to posterior dimension and the lateral dimension may vary based on the size of the relevant target site. The length of the implant may also be selected to match the surgeon's preference for the spacing of the implant on/in the spine. In general, the length of implants of the present disclosure (for bone fastener/screw not including towers) range from between about 20 mm and about 90 mm and often about 25 mm to about 60 mm, with outer diameters from between about 3.5 mm and about 8.5 mm, possibly as much as 10 mm, and with minor diameters of between about 2.0 mm and about 7.5 mm.

Deployment Tools.

While the particulars of the tools for deployment of the implants are beyond the focus of this application, the implant deployment tools include drills, drill guides, taps, (screw) drivers, insertion tools, extraction tools, or tools that may be used for both insertion and extraction. Yet another advantage of the device systems as disclosed herein is that a tower may be disassembled and removed individually if a tower is not able to be removed in a regular manner.

Multi-Level Surgery

For convenience, the description set forth above provides therapy to fixation of non-specified motion segment(s) (i.e., one disc space between two adjacent vertebrae), one of skill in the art will recognize that the process set forth above may applied to constructs so that more than one motion segment, in multiple spinal levels (e.g., lumbar; thoracic; cervical) may receive therapy (such as fusion) during a single surgical intervention.

Open Surgery.

While the focus of this disclosure has been on a minimally invasive posterior access and therapies, the various implants described in this application may be used with other access routes including an open approach.

Kits.

One of skill in the art will recognize that the surgical procedures set forth above may benefit from various kits of tools and components for use in these procedures. Kits may focus on reusable or disposable components for creating an access route. Other kits may focus on the tools for preparing the targeted surgical site(s). A kit may include many (possibly even all) the components necessary for a particular procedure including the components needed to create the access route, prepare the targeted sites and even an assortment of implants, as well as the instruments needed for their deployment.

One of skill in the art will recognize that some of the alternative implementations set forth above are not universally mutually exclusive and that in some cases additional implementations can be created that employ aspects of two or more of the variations described above. Likewise, the present disclosure is not limited to the specific examples or particular examples provided to promote understanding of the various teachings of the present disclosure. Moreover, the scope of the claims which follow covers the range of variations, modifications, and substitutes for the components described herein as would be known to those of skill in the art. Individual claims may be tailored to claim particular examples out of the array of examples disclosed above. Some claims may be tailored to claim alternative examples rather than preferred examples. Some claims may cover an example set forth above with a modification from another example as the present disclosure does not include drawings of all possible combinations of feature sets.

The legal limitations of the scope of the claimed invention are set forth in the claims that follow and extend to cover their legal equivalents. Those unfamiliar with the legal tests for equivalency should consult a person registered to practice before the patent authority which granted this patent such as the United States Patent and Trademark Office or its counterpart

What is claimed is:

1. A pedicle screw system comprising:
a bone fastener having a distal portion adapted to be inserted into bone and a proximal portion having an enlarged head;
a receiving member having an bottom portion and a top portion and defining a bore extending from the bottom portion to the top portion and defining an a lower opening in the bottom portion, a recess at the bottom portion for receiving the enlarged head of the bone fastener, and an upper opening in the top portion, the receiving member further defining a pair of U-shaped channels having openings at the top portion;
a rod extending across the U-shaped channels;
a lower member positioned within the bore of the receiving member above the enlarged head of the bone fastener and below the rod, the lower member comprising at least one extension that extends upwardly alongside the rod; and
a fixation member positioned in the bore within top portion, the fixation member comprising an external thread or cam surface that engages an internal thread or cam surface formed on an inner surface of the receiving member, fixation member configured such that rotation of the fixation member causes the fixation member to push down on the extension of the lower member and on the rod to lock the rod against the receiving member and the enlarged head against the recess of the receiving member.

2. An insertion system coupling a spinal rod to a pedicle screw system; the system comprising:
a first bone fastener configured to be coupled to a first vertebrae, the first bone fastener comprising a receiving member comprising a top portion with a pair of channels having an opening at the top portion, the top portion including at least one engagement member on each side of the top portion separated by the openings of the pair of channels, the first bone fastener including an outer lip at the top portion;
an outer sleeve having a proximal end and a distal end, the distal end being configured to engage the top portion of the first bone fastener and the outer lip;
at least one translating blade extending within the sleeve, wherein the at least one blade comprises proximal and distal ends, the distal end of each blade having an engagement member configured to engage a corresponding engagement member on the top portion of the receiving member;
a coupling member configured to hold the outer sleeve; and
a translating member configured to translate the at least one translating blade.

3. A method of implanting a spinal stabilization system, the method comprising:
engaging a distal end of a placement device with an upper portion of a first bone fastener wherein the upper portion of the bone fastener comprises a pair of structural members defining a channel, the placement device comprising a channel extending from a proximal end of the placement device to the distal end of the placement device, wherein the channel of the placement device is aligned with the channel of the structural members;
translating engagement members in the placement device into corresponding engagement members on the upper portion of the first bone fastener;
advancing the placement device and the first bone fastener through an incision towards a first vertebral body;
coupling the first bone fastener to the first vertebral body; and
advancing a rod through the channel of the placement device and between the pair of structural members towards the first bone fastener.
